(19) United States
(12) Patent Application Publication
(10) Pub. No.: US 2023/0189844 A1
(43) Pub. Date: Jun. 22, 2023

(54) PROTEASE ANIMAL FEED FORMULATION

(71) Applicant: Novozymes A/S, Bagsvaerd (DK)

(72) Inventor: Flemming Borup, Tygelsjoe (SE)

(73) Assignee: Novozymes A/S, Bagsvaerd (DK)

(21) Appl. No.: 18/063,501

(22) Filed: Dec. 8, 2022

(30) Foreign Application Priority Data
Dec. 16, 2021 (EP) 21215252.4
Dec. 16, 2021 (EP) 21215253.2
Dec. 16, 2021 (EP) 21215258.1
Dec. 16, 2021 (EP) 21215262.3

(51) Int. Cl. A23K 20/189; A23K 50/75; A23K 50/30; A23K 20/147; A23K 10/14; A23K 20/174; A23K 40/10; C12N 9/48

(52) U.S. Cl. CPC A23K 20/189; A23K 10/14; A23K 20/147; A23K 20/174; A23K 40/10; A23K 50/30; A23K 50/75; C12N 9/48

(57) ABSTRACT

The reformulation of RONOZYME® ProAct protease polypeptide to a more economical microgranulate, extrudate, high-shear granulate or spray drying formulation each provide good thermostability and suitability for use as an animal feed additive.

### REFERENCE TO SEQUENCE LISTING

This application contains a Sequence Listing in computer readable form. The name of the file containing the Sequence Listing is SQ.XML, which was created on Dec. 8, 2022, and contains 4,523 bytes. The computer readable form is incorporated herein by reference.

### FIELD OF THE INVENTION

The present invention relates to novel granule formulations of a protease for use in animal feed.

### BACKGROUND OF THE INVENTION

The prospects of climatic changes, linked to a loss of fertile farmland and thus a lower agricultural production, force the producers to look for more effective ways of food production. Steadily increasing prices of traditional feed commodities and the stagnating purchase prices of meat on the other, means that farmers are constantly looking for ways to reduce their production costs.

Pelleting was introduced in the mid-1920′s to the feed industry to improve feed utilization and improve handling characteristics. The early pelleting process involved mixing the feed ingredients and pelleting them with no further treatment. The rationale for this approach was to prevent alterations to vitamins and proteins due to the addition of heat to the feed mix. The focus on research into the pelleting process since the 1960′s has been on improving the conditioning operation, with emphasis on increasing the retention time and increasing the temperature to which the mash is conditioned.

Animal feed comprising enzymes is known to have several advantages depending on the enzymes used. Typically, the animal feed is found in one of two forms: mash feed composed of all diet components mixed together or pelleted feed where the different diet components are compressed down into pellets with roughly the same size. Pelleted feed is often advantageous for several reasons such as the availability of all needed ingredients and easy storage and handling.

Feed pellets may include one or more enzymes and are typically produced by mixing granules comprising the active ingredients such as enzymes with other ingredients such as e.g. cereals and nutrients, followed by conditioning and processing of the mixture into pellets. It is important that nutrients and enzymes are evenly distributed in the feed to ensure that all animals receive an optimal blend of nutrients and enzymes via the feed.

During the conditioning and pelleting process, the temperature is increased and can in some instances reach high temperatures. Furthermore, the high temperatures during the conditioning and pelleting process may negatively affect the stability of the enzyme and thus the activity thereof. Formulation of the enzymes prior to pelleting is a judicious selection process wherein a formulation which ensures the enzyme activity after the pelleting process, transportation and long-term storage.

Granulation of enzymes is a difficult task. In spite of the fact that patent applications on different methods for the production of granulated and dust-free enzymes have been numerous, hardly more than two or three different granulation methods are in use today on an industrial scale. The most common among those methods are: Embedding of the enzymes into spheres of a waxy material by a so-called prilling process, vide German DOS No. 2,060,095, and the process described in British Patent Specification No. 1,362,365, where the enzyme is mixed with a filler, a binder and water, whereafter it is extruded and spheronized. By these two methods enzyme granules with very low dust level can be produced. However, both of these methods have some drawbacks. In the prilling process at least about 50 percent of the product must be a waxy material, for example an ethoxylated fatty alcohol, which is rather expensive. The other method mentioned above has the drawback that the production on an industrial scale is difficult.

The commercially available product RONOZYME® ProAct is available in a heat stable free-flowing & dust free CT formulation or in a liquid form (L) for post-pelleting liquid applications. This formulation involves a core wherein the enzyme is absorbed and an extra coating to ensure the stability of the costly enzyme during pelleting process, transportation and long-term storage. RONOZYME® ProAct is a top performing product within animal feed. Both formulations are intended to be mixed into premixtures and/or feeding stuffs to obtain a minimum enzyme activity levels of 15 000 PROT/kg in feeding stuffs for chickens for fattening. The CT formulation, a granulated coated thermo-tolerant form, at least in part, makes RONOZYME® ProAct the most stable feed protease. RONOZYME® ProAct is stable throughout the intestinal tract and supplements the performance of other feed enzymes such as carbohydrases and phytases. RONOZYME® ProAct has outstanding stability in all feed applications, including premixes and pelleted feeds. The dust-free formulation ensures there are no safety issues while being incorporated in feed.

Proteases are widely used in a variety of applications, including detergents, textiles, baking and animal feed. These applications generally benefit from enzymes that are protected from moisture, temperature, and harsh chemicals. Accordingly, the enzyme is generally granulated and coated with one or more protective coatings.

However, granulation and coating add significant costs to enzyme products. There is a need to provide an enzyme granule with low cost.

### SUMMARY OF THE INVENTION

The invention provides novel granules or formulations of the polypeptide of RONOZYME® ProAct and variants thereof. It has surprisingly been found that the polypeptide of RONOZYME® ProAct and variants thereof can be formulated in much cheaper and traditionally less robust formulations and maintain approximately the same level of activity after conditions which accurately replicate pelleting conditions.

The novel granules and formulations of the protease increases digestibility of protein and ensures more amino acids are available to the animal. The amount of nitrogen excreted is decreased. Ultimately this can increase the opportunity to use cheaper feed materials and so reduce feed costs. Alternatively, the protein content in the diet can be reduced while still maintaining animal performance

An aspect of the invention is directed to an animal feed additive comprising a polypeptide having protease activity, wherein the polypeptide has at least 70% sequence identity to SEQ ID NO:1; characterized in that the enzyme is formulated in a formulation selected from the group consisting of:
i. a granule prepared by an extrusion process;
ii. a granule prepared by a spray-drying process;
iii. a granule comprising a salt core, such as a sodium sulfate or sodium chloride core, and a protease-containing layer; and
iv. a granule prepared by a high-shear granulation process

A further aspect of the invention is directed to a granule, comprising a salt core, such as a sodium sulfate or sodium chloride core, and protease, typically an acid-stable protease containing layer, wherein the protease is a polypeptide having protease activity and having at least 70% sequence identity with SEQ ID NO: 1. The granule comprising a salt core and a protease containing layer is typically a microgranule.

A still further aspect of the invention is directed to a granule comprising a polypeptide having protease activity and having at least 70% sequence identity to the polypeptide of SEQ ID NO: 1; said granule prepared by a spray-drying process.

An aspect of the invention is directed to a granule comprising a polypeptide having protease activity and having at least 70% sequence identity to the polypeptide of SEQ ID NO: 1, said granule prepared by an extrusion process. One novel formulation of a polypeptide having protease activity and having at least 70% sequence identity to the polypeptide of SEQ ID NO: 1 is prepared by extruding technology or by an extrusion process. It advantageously is less costly to prepare and surprisingly allows for suitable stability to the polypeptide for use as an animal feed additive. Surprisingly, the protein denaturation typically associated with extruding technology processes, is not observed to a significant degree when using the polypeptide of RONOZYME® ProAct.

One aspect of the invention is directed to an animal feed additive comprising extruded enzyme pellets wherein said enzyme is a polypeptide having protease activity and having at least 70% sequence identity to SEQ ID NO:1. According to an aspect of the invention, the method of the present invention comprises (a) combining a polypeptide having protease activity, a solid carrier, optionally water, and a meltable hydrophobic substance to provide a combined product; (b) optionally applying sufficient heat to the combined product to allow the hydrophobic substance to melt; (c) extruding the product of step (b); and (d) allowing the extruded product of step (c) to dry and cool or actively drying and cooling the extruded product of step (c) to provide the thermostable enzyme product, wherein the polypeptide having protease activity has at least 70% sequence identity to SEQ ID NO: 1, namely at least 75% sequence identity to the polypeptide of RONOZYME® ProAct. A further aspect of the invention is directed to a method of preparing an animal feed additive comprising a polypeptide having protease activity having at least 70% sequence identity to SEQ ID NO: 1, namely having at least 75% sequence identity to the polypeptide of RONOZYME® ProAct, comprising an extrusion process, said process comprising extruding a combination comprising said polypeptide, a meltable hydrophobic substance, and a solid carrier. In another embodiment, the present invention encompasses a method for preparing a thermostable enzyme product for use in the manufacture of animal feed comprising (a) combining an enzyme, a solid carrier and a meltable hydrophobic substance to provide a combined product; (b1) reducing the moisture content by applying heat to the combined product and (b2) melting the hydrophobic substance; and (c) cooling the combined product to provide the thermostable enzyme product, wherein the thermostable enzyme is the polypeptide having protease activity and having at least 70% sequence identity to SEQ ID NO: 1.

In still another embodiment, the present invention encompasses a method for preparing a thermostable enzyme product for use in the manufacture of animal feed comprising (a) combining an enzyme, a solid carrier, a meltable hydrophobic substance to provide a combined product and optionally additional water to form a suitable paste; (b) optionally applying sufficient heat to the combined product to allow the hydrophobic substance to melt; (c) extruding the product of step (b); and (d) drying and cooling the extruded product of step (c) to provide the thermostable enzyme product. In one aspect of this embodiment, the meltable hydrophobic substance is added in step (a) as solid flakes or as a pre-melted molten liquid. The skilled person will recognize that if the meltable hydrophobic substance is added as a pre-melted molten liquid, step (b) may not be necessary. The components referred to in step (a) may be combined in a single step or alternatively, in separate steps. For example, the enzyme may first be combined with the solid carrier and optionally water, optionally dried, and then the resulting enzyme/carrier combination combined with the meltable hydrophobic substance.

A further aspect of the invention is directed to a granule comprising a polypeptide having protease activity and having at least 70% sequence identity to the polypeptide of SEQ ID NO: 1, said granule prepared by a high-shear granulation process. A related aspect of the invention is directed to a use of a granule defined by the invention in an animal feed or for the preparation of an animal feed.

A further aspect of the invention is directed to a method of preparing a granule comprising a polypeptide having protease activity and having at least 70% sequence identity to the polypeptide of SEQ ID NO: 1, said method comprising a process comprising a formulation process selected from the group consisting of
i. an extrusion process;
ii. a spray-drying process;
iii. spraying or wetting a salt core with a protease-containing liquid; and
iv. a high-shear granulation process.

In still another embodiment, the present invention encompasses a method for preparing a thermostable enzyme product for use in the manufacture of animal feed comprising (a) combining a polypeptide having protease activity and having at least 70% sequence identity to SEQ ID NO: 1, a solid carrier, a meltable hydrophobic substance to provide a combined product and optionally additional water to form a suitable paste; (b) melting the hydrophobic substance, or allowing the hydrophobic to melt, optionally by applying heat to the combined product; (c) extruding the product of step (b); and (d) optionally drying and cooling the extruded product of step (c) to provide the thermostable enzyme product.

In one aspect of this embodiment, the meltable hydrophobic substance is added in step (a) as solid flakes or as a pre-melted molten liquid. The skilled person will recognize that if the meltable hydrophobic substance is added as a pre-melted molten liquid, step (b) may not be necessary. The components referred to in step (a) may be combined in a single step or alternatively, in separate steps. For example, the enzyme may first be combined with the solid carrier and optionally water, optionally dried, and then the resulting enzyme/carrier combination combined with the meltable hydrophobic substance.

The invention provides a novel formulation of the polypeptide of RONOZYME® ProAct. The novel formulation is prepared by high-shear granulation. It advantageously is less costly to prepare and surprisingly allows for suitable stability to the polypeptide for use as an animal feed additive.

An aspect of the invention is directed to an enzyme granulate said granulate prepared by a method comprising a high-shear granulation process, said granulate comprising a polypeptide having protease activity, said polypeptide having at least 70% sequence identity with a polypeptide of SEQ ID NO:1. The granulate suitably further comprises at least one binder and cellulose or a derivative thereof.

A further aspect of the invention is directed to an animal feed additive comprising the enzyme granulate of the invention. A further aspect of the invention is directed to an animal feed comprising the enzyme granulate of the invention. A further aspect of the invention is directed to an animal feed comprising the animal feed additive of the invention. A further aspect of the invention is directed to a method of preparing a granulate comprising a granulate comprising a high-shear granulation, said high-shear granulation process comprising
A. forming a powder mixture by combining at least
   i. cellulose or a derivative thereof
   ii. optionally a binder; and
   iii. optionally a filler; and
B. adding a liquid phase granulating agent
   wherein the polypeptide having protease activity is added to either the powder mixture or to the liquid phase granulating agent and wherein the at least one binder is added to either the powder mixture or to the liquid phase granulating agent or both; wherein said polypeptide having protease activity is a polypeptide having at least 70% sequence identity to SEQ ID NO:1,
   or wherein said high-shear granulation process comprises
A′. forming a powder mixture by combining at least
   i. cellulose or a derivative thereof
   ii. a binder; and
   iii. optionally a filler; and
B′. adding a liquid phase granulating agent
   wherein the polypeptide having protease activity is added to either the powder mixture or to the liquid phase granulating agent, wherein said polypeptide having protease activity is a polypeptide having at least 70% sequence identity to SEQ ID NO:1.

### DETAILED DESCRIPTION OF THE INVENTION

An aspect of the invention is directed to an animal feed additive comprising a polypeptide having protease activity, wherein the protease comprises a polypeptide having at least 70% sequence identity to SEQ ID NO:1; characterized in that the protease is formulated in a formulation selected from the group consisting of:
i. a granule prepared by an extrusion process;
ii. a granule prepared by a spray-drying process;
iii. a granule comprising a salt core, such as a sodium sulfate or sodium chloride core, and a protease-containing layer; and
iv. a granule prepared by a high-shear granulation process.

A further aspect of the invention is directed to a granule comprising a polypeptide having protease activity, wherein the protease comprises a polypeptide having at least 70% sequence identity to SEQ ID NO:1 characterized in that the granule is selected from the group consisting of a i. comprising a salt core, such as a sodium sulfate or sodium chloride core, and a protease-containing layer; ii. a granule prepared by a spray-drying process; a granule prepared by an extrusion process; and granule prepared by a high-shear granulation process.

It has surprisingly been found that SEQ ID NO:1 and variants thereof are stable enough to be used as an animal feed additive when formulated as a granule selected from the group consisting of a microgranule comprising a salt core and a protease-containing layer, a granule prepared by a spray-drying process, a granule prepared by an extrusion process; and granule prepared by a high-shear granulation process. Furthermore, it has been surprisingly found that these novel inexpensive granules of SEQ ID NO:1 and variants thereof are equally stable to the commercially available RONOZYME® ProAct CT which comprises a more robust formulation.

### Definitions

Animal: The term “animal” refers to all animals except humans. Examples of animals are non-ruminants, and ruminants. Ruminant animals include, for example, animals such as sheep, goats, cattle, e.g. beef cattle, cows, and young calves, deer, yank, camel, llama and kangaroo. Non-ruminant animals include mono-gastric animals, e.g. pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

Animal feed: The term “animal feed” refers to any compound, preparation, or mixture suitable for, or intended for intake by an animal. Animal feed for a mono-gastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Body Weight Gain: The term “body weight gain” means an increase in live weight of an animal during a given period of time e.g. the increase in weight from day 1 to day 21.

Composition: The term “composition” refers to a composition comprising a carrier and at least one enzyme of the present invention. The compositions described herein may be mixed with an animal feed and referred to as a “mash feed.”

Concentrates: The term “concentrates” means feed with high protein and energy concentrations, such as fish meal, molasses, oligosaccharides, sorghum, seeds and grains (either whole or prepared by crushing, milling, etc from e.g. corn, oats, rye, barley, wheat), oilseed press cake (e.g. from cottonseed, safflower, sunflower, soybean, rapeseed/canola, peanut or groundnut), palm kernel cake, yeast derived material and distillers grains (such as wet distillers grains (WDS) and dried distillers grains with solubles (DDGS)).

Direct Fed Microbial: The term “direct fed microbial” means live micro-organisms including spores which, when administered in adequate amounts, confer a benefit, such as improved digestion or health, on the host.

Effective amount/concentration/dosage: The terms “effective amount”, “effective concentration”, or “effective dosage” are defined as the amount, concentration, or dosage of the enzyme sufficient to improve the digestion or yield of an animal. The actual effective dosage in absolute numbers depends on factors including: the state of health of the animal in question, other ingredients present. The “effective amount”, “effective concentration”, or “effective dosage” of the enzyme may be determined by routine assays known to those skilled in the art.

Extruding (expansion) technology is a technology in modern feed processing. Feed processed by extrusion technology has many properties, wanted and unwanted, such as starch gelatinization and degradation, protein denaturation, reducing anti-nutritional factors, increasing palatability, etc. In the extrusion technology, material containing a certain moisture level is fed into the feed extruder, driven by the screw rod and screw, whereby material moves forward to form an axial direction. The material and screw, material and barrel as well as the material inside generate friction, so that material is strongly extruded, stirred and sheared, makes the material further refines and homogeneous, with the increasing pressure and temperature in the feed extruder machine chamber and the internal friction between the material and screw, material and barrel. With the increase of temperature, high pressure and high shear force, the composition of materials has undergone complex physical and chemical changes. Finally, the paste material is ejected from the die hole, which produces instantaneous pressure difference, and the material is expanded, thus forming a loose, porous and crisp extruded product.

Feed Conversion Ratio: The term “feed conversion ratio” the amount of feed fed to an animal to increase the weight of the animal by a specified amount. An improved feed conversion ratio means a lower feed conversion ratio. By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Nutrient Digestibility: The term “nutrient digestibility” means the fraction of a nutrient that disappears from the gastro-intestinal tract or a specified segment of the gastro-intestinal tract, e.g. the small intestine. Nutrient digestibility may be measured as the difference between what is administered to the subject and what. comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed. Nutrient digestibility as used herein encompasses starch digestibility, fat digestibility, protein digestibility, and amino acid digestibility.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

Pellet: The terms “pellet” and/or “pelleting” refer to solid rounded, spherical and/or cylindrical tablets or pellets and the processes for forming such solid shapes, particularly feed pellets and solid extruded animal feed. As used herein, the terms “extrusion” or “extruding” are terms well known in the art and refer to a process of forcing a composition, as described herein, through an orifice under pressure.

Poultry: The term “poultry” means domesticated birds kept by humans for the eggs they produce and/or their meat and/or their feathers. Poultry includes broilers and layers. Poultry include members of the superorder Galloanserae (fowl), especially the order Galliformes (which includes chickens, Guineafowls, quails and turkeys) and the family Anatidae, in order Anseriformes, commonly known as “waterfowl” and including domestic ducks and domestic geese. Poultry also includes other birds that are killed for their meat, such as the young of pigeons. Examples of poultry include chickens (including layers, broilers and chicks), ducks, geese, pigeons, turkeys and quail.

Roughage: The term “roughage” means dry plant material with high levels of fiber, such as fiber, bran, husks from seeds and grains and crop residues (such as stover, copra, straw, chaff, sugar beet waste).

Ruminant: The term “ruminant” means a mammal that digests plant-based food by initially fermenting/degrading it within the animal’s first compartment of the stomach, principally through bacterial actions, then regurgitating the semi-digested mass, now known as cud, and chewing it again. The process of re-chewing the cud to further break down plant matter and stimulate digestion is called “ruminating”. Examples of ruminants are cattle, cow, beef cattle, young calf, goat, sheep, lamb, deer, yank, camel and llama.

Sequence Identity: The relatedness between two amino acid sequences is described by the parameter “sequence identity”. Sequence identity is determined by either of the following three methods:

### Sequence Identity Determination Method 1

The sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), version 6.6.0. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

$$\begin{matrix}\left(\text{Identical Residues} \times \text{100}\right) / \left(\text{Length of Alignment} - \text{Total Number}\right) \\ o f \text{Gaps in} \left(\text{Alignment}\right)\end{matrix}$$

### Sequence Identity Determination Method 2

The sequence identity between two amino acid sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), version 6.6.0. The parameters used are a gap open penalty of 10, a gap extension penalty of 0.5, and the EBLOSUM62 (EMBOSS version of BLOSUM62) substitution matrix. The percent sequence identity is calculated as follows:

$$\begin{matrix}\left(\text{Identical Residues} \times \text{100}\right) / \left(\text{Length of the Shortest Sequence}\right) \\ \text{in the} \left(\text{Alignment}\right)\end{matrix}$$

### Sequence Identity Determination Method 3

The sequence identity between two amino acid sequences is determined using Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), version 6.6.0. The parameters used are a gap open penalty of 10, a gap extension penalty of 0.5, and the EBLOSUM62 (EMBOSS version of BLOSUM62) substitution matrix. The percent identity is calculated as follows:

$$\left(\text{Identical Residues} \times \text{100}\right) / \left(\text{Length of the Alignment}\right)$$

Silage: The term “silage” means fermented, high-moisture stored fodder which can be fed to ruminants (cud-chewing animals such as cattle and sheep) or used as a biofuel feedstock for anaerobic digesters. It is fermented and stored in a process called ensilage, ensiling or silaging, and is usually made from grass or cereal crops (e.g. maize, sorghum, oats, rye, timothy etc forage grass plants),) or legume crops like clovers/trefoils, alfalfa, vetches, using the entire green plant (not just the grain). Silage can be made from many field crops, and special terms may be used depending on type (oatlage for oats, haylage for alfalfa). Silage is made either by placing cut green vegetation in a silo, by piling it in a large heap covered with plastic sheet, or by wrapping large bales in plastic film.

Particle Size Distribution (PSD): The term “Particle Size Distribution” or “PSD” is herein used for granules of the invention and defines the relative amount, typically by volume, of particles present according to size. The PSD is described as the D-Values D10, D50 and D90, wherein D10 refers to the 10% percentile of the particle size distribution (meaning that 10% of the volume of the particles has a size equal or less than the given value), D50 describes the 50% percentile and D90 describes the 90% percentile. Particle size distribution may be measured using laser diffraction methods or optical digital imaging methods or sieve analysis. D-Values reported herein were measured by laser diffraction, where the particle size was reported as a volume equivalent sphere diameter.

Small enzyme granule: The term “small enzyme granule” refers to a granule containing an enzyme with a median size (diameter) of around 100-2000 micrometers, preferably 200-1500 micrometers, more preferably 300-1200 micrometers.

Swine: The term “swine” or “pigs” means domesticated pigs kept by humans for food, such as their meat. Swine includes members of the genus Sus, such as Sus scrofa domesticus or Sus domesticus and include piglets, growing pigs, and sows.

Thermostable: The term “thermostable” is a term that is known in the art, and in a preferred aspect, stable is intended to mean the ability of the enzyme to remain active under thermal stress, such as during the extrusion process. In relation to the polypeptide having protease activity, thermostability is intended to mean that the protease in the extrusion product maintains at least 60% of the 75.000 prot/g activity, such as at least 65% of the 75.000 prot/g activity, such as at least 70% of the 75.000 prot/g activity, such as at least 75% of the 75.000 prot/g activity, such as at least 80% of the 75.000 prot/g activity, such as at least 85% of the 75.000 prot/g activity, such as at least 90% of the 75.000 prot/g activity, such as at least 95% of the 75.000 prot/g activity of the polypeptide having protease activity of SEQ ID NO:1.

Vegetable protein: The term “vegetable protein” refers to any compound, preparation or mixture that includes at least one protein derived from or originating from a vegetable, including modified proteins and protein-derivatives.

According to an aspect of the invention, the method of the present invention comprises (a) combining a polypeptide having protease activity, a solid carrier, optionally water, and a meltable hydrophobic substance to provide a combined product; (b) optionally applying sufficient heat to the combined product to allow the hydrophobic substance to melt; (c) extruding the product of step (b); and (d) allowing the extruded product of step (c) to dry and cool or actively drying and cooling the extruded product of step (c) to provide the thermostable enzyme product, wherein the polypeptide having protease activity has at least 70% sequence identity to SEQ ID NO: 1, namely at least 75% sequence identity to the polypeptide of RONOZYME® ProAct.

### The Polypeptide

Polypeptides having protease activity, or proteases, are sometimes also designated peptidases, proteinases, peptide hydrolases, or proteolytic enzymes. Proteases may be of the exo-type that hydrolyse peptides starting at either end thereof, or of the endo-type that act internally in polypeptide chains (endopeptidases). Endopeptidases show activity on N- and C-terminally blocked peptide substrates that are relevant for the specificity of the protease in question.

Protease activity can be measured using any assay, in which a substrate is employed, that includes peptide bonds relevant for the specificity of the protease in question. Assay-pH and assay-temperature are likewise to be adapted to the protease in question. Examples of assay-pH-values are pH 5, 6, 7, 8, 9, 10, or 11. Examples of assay-temperatures are 30, 35, 37, 40, 45, 50, 55, 60, 65 or 70, 80, 90, or 95° C.

Examples of protease substrates are casein, and pNA-substrates, such as Suc-AAPF-NA (available e. g. from Sigma S7388). The capital letters in this pNA-substrate refers to the one-letter amino acid code. Another example is Protazyme AK (azurine-dyed crosslinked casein prepared as tablets by Megazyme T-PRAK). For pH-activity and pH-stability studies, the pNA-substrate is preferred, whereas for temperature activity studies, the Protazyme AK substrate is preferred.

For the purpose of the present invention, protease activity was determined using assays which are described in in the art, such as the Suc-AAPF-pNA assay, Protazyme AK assay, Suc-AAPX-pNA assay and o-Phthaldialdehyde (OPA). For the Protazyme AK assay, insoluble Protazyme AK (Azurine-Crosslinked Casein) substrate liberates a blue colour when incubated with the protease and the colour is determined as a measurement of protease activity. For the Suc-AAPF-pNA assay, the colourless Suc-AAPF-pNA substrate liberates yellow paranitroaniline when incubated with the protease and the yellow colour is determined as a measurement of protease activity.

The granulate comprises a polypeptide having protease activity, said polypeptide having at least 70% sequence identity with a polypeptide of SEQ ID NO:1, as defined herein:

SEQ ID NO: 1

Ala Asp Ile Ile Gly Gly Leu Ala Tyr Thr Met Gly Gly Arg Cys Ser
Val Gly Phe Ala Ala Thr Asn Ala Ala Gly Gln Pro Gly Phe Val Thr
Ala Gly His Cys Gly Arg Val Gly Thr Gln Val Thr Ile Gly Asn Gly
Arg Gly Val Phe Glu Gln Ser Val Phe Pro Gly Asn Asp Ala Ala Phe
Val Arg Gly Thr Ser Asn Phe Thr Leu Thr Asn Leu Val Ser Arg Tyr
Asn Thr Gly Gly Tyr Ala Thr Val Ala Gly His Asn Gln Ala Pro Ile
Gly Ser Ser Val Cys Arg Ser Gly Ser Thr Thr Gly Trp His Cys Gly
Thr Ile Gln Ala Arg Gly Gln Ser Val Ser Tyr Pro Glu Gly Thr Val
Thr Asn Met Thr Arg Thr Thr Val Cys Ala Glu Pro Gly Asp Ser Gly
Gly Ser Tyr Ile Ser Gly Thr Gln Ala Gln Gly Val Thr Ser Gly Gly
Ser Gly Asn Cys Arg Thr Gly Gly Thr Thr Phe Tyr Gln Glu Val Thr
Pro Met Val Asn Ser Trp Gly Val Arg Leu Arg Thr

The polypeptide may be natural or synthetic. It is suitably obtained, obtainable from Nocardiopsis sp. NRRL 18262. It is suitably derivable from a polypeptide obtained from Nocardiopsis sp. NRRL 18262.

Ronozyme ProAct is a preparation of serine protease produced by a genetically modified strain of Bacillus licheniformis. It is produced by fermentation of a sporulation deficient Bacillus licheniformis strain Rh 3 which expresses a synthetic gene encoding a serine protease (EC 3.4.21.-). Accordingly, in one aspect of the invention, the polypeptide having protease activity is produced by a genetically modified strain of Bacillus licheniformis, preferably a sporulation deficient Bacillus licheniformis strain Rh 3, and has least 70% sequence identity to a polypeptide having SEQ ID NO.1.

Typically, the polypeptide having protease activity has at least 70% sequence identity with a polypeptide of SEQ ID NO:1 and has protease activity, such as at least 75% sequence identity with a polypeptide of SEQ ID NO:1, such as at least 80% sequence identity with a polypeptide of SEQ ID NO:1,such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100%.

In a further typical embodiment, the polypeptide having protease activity comprises a polypeptide sequence having at least 70% sequence identity with a polypeptide of SEQ ID NO:1 and further comprises an N-terminal sequence 1 to 30 amino acid residues and/or a C-terminal sequence of 1 to 30 amino acid residues. The polypeptide having protease activity may comprises a polypeptide sequence having at least 75% sequence identity with a polypeptide of SEQ ID NO:1 such as at least 75% sequence identity with a polypeptide of SEQ ID NO:1, such as at least 80% sequence identity with a polypeptide of SEQ ID NO:1,such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100%, and further comprises an N-terminal sequence 1 to 30 amino acid residues and/or a C-terminal sequence of 1 to 30 amino acid residues.

The polypeptide having protease activity typically is selected from a polypeptide having at least 75%, such as at least 80%, such as at least 85%, preferably at least 90%, such as at least 95%, such as at least 96%, such as at least 97%, such as at least 98%, such as at least 99%, such as 100% sequence identity to SEQ ID NO:1, SEQ ID NO:2, or SEQ ID NO:3. The polypeptide having protease activity is typically selected from the group consisting of
i. an amino acid sequence having at least 80% sequence identity to SEQ ID NO:1;
ii. an amino acid sequence having at least 80% sequence identity to SEQ ID NO:2; and
iii. an amino acid sequence having at least 80% sequence identity to SEQ ID NO:3

SEQ ID NO:2:

ADIIGGLAYT IGGRCSVGFA ATNAAGQPGF VTAGHCGRVG TQVTIGNGRG VFEQSVFPGN
DAAFVRGTSNFTLTNLVSRY NTGGYATVAG HNQAPIGSSV CRSGSTTGWH CGTIQARGQS
VSYPEGTVTNMTRTTVCAEP GDSGGSYISG TQAQGVTSGG SGNCRTGGTT FYQEVTPMVN
SWGVRLRT

SEQ ID NO: 3:

MKKPLGKIVASTALLISVAFSSSIASAAPAPVPQTPVADDSAASMTEALKRDLDLTSAEAEELLSA
QEAAIETDAEATEAAGEAYGGSLFDTETLELTVLVTDASAVEAVEATGAQATVVSHGTEGLTEV
VEDLNGAEVPESVLGWYPDVESDTVVVEVLEGSDADVAALADAGVDSSSVRVEEAEEAPQVY
ADIIGGLAYT MGGRCSVGFA ATNAAGQPGF VTAGHCGRVG TQVTIGNGRG VFEQSVFPGN
DAAFVRGTSN FTLTNLVSRY NTGGYATVAG HNQAPIGSSV CRSGSTTGWH CGTIQARGQS
VSYPEGTVTN MTRTTVCAEP GDSGGSYISG TQAQGVTSGG SGNCRTGGTT
FYQEVTPMVN SWGVRLRT QSHVQSAP

The polypeptide having protease activity typically has minimum protease activity levels of at least 35.000 PROT/kg, such as at least 50.000 PROT/kg, such as at least 75.000 PROT/kg. In relation to the polypeptide having protease activity, thermostability is intended to mean that the protease in the extrusion product maintains at least 60% of the 75.000 prot/g activity, such as at least 65% of the 75.000 prot/g activity, such as at least 70% of the 75.000 prot/g activity, such as at least 75% of the 75.000 prot/g activity, such as at least 80% of the 75.000 prot/g activity, such as at least 85% of the 75.000 prot/g activity, such as at least 90% of the 75.000 prot/g activity, such as at least 95% of the 75.000 prot/g activity of the polypeptide having protease activity of SEQ ID NO:1.

Ronozyme ProAct is a preparation of serine protease produced by a genetically modified strain of Bacillus licheniformis. It is produced by fermentation of a sporulation deficient Bacillus licheniformis strain Rh 3 which expresses a synthetic gene encoding a serine protease (EC 3.4.21.-). Accordingly, in one aspect of the invention, the polypeptide having protease activity is produced by a genetically modified strain of Bacillus licheniformis, preferably a sporulation deficient Bacillus licheniformis strain Rh 3, and has least 70% sequence identity to a polypeptide having SEQ ID NO:1. In a typically embodiment, the polypeptide of the invention has at least 60% sequence identity to SEQ ID NO:1.

### An Acid-Stable Protease

In a preferred embodiment, the protease is an acid-stable protease. In the present context, the term acid-stable means, that the protease activity of the pure protease enzyme, in a dilution corresponding to A280 = 1.0, and following incubation for 2 hours at 37° C. in the following buffer:
100 mM succinic acid, 100 mM HEPES, 100 mM CHES,
· 100 mM CABS, 1 mM CaCl2, 150 mM KCI, 0.01% TritonX-100, pH 3.5,
is at least 40% of the reference activity, as measured using the assay described in pH-stability assay herein (substrate: Suc-AAPF-pNA, pH 9. 0, 25° C.).

In particular embodiments of the above acid-stability definition, the protease activity is at least 45, 50, 55, 60, 65, 70, 75, 80, 85, 90, 95, or at least 97% of the reference activity.

The term reference activity refers to the protease activity of the same protease, following incubation in pure form, in a dilution corresponding to A280 = 1.0, for 2 hours at 5° C. in the following buffer: 100 mM succinic acid, 100 mM HEPES, 100 mM CHES, 100 mM CABS, 1 mM CaCl2, 150 mM KCI, 0.01% TritonDX-100, pH 9.0, wherein the activity is determined as described above.

In other words, the method of determining acid-stability comprises the following steps:
a) The protease sample to be tested (in pure form, A280 = 1.0) is divided in two aliquots (I and II);
b) Aliquot I is incubated for 2 hours at 37° C. and pH 3.5;
c) Residual activity of aliquot I is measured (pH 9.0 and 25° C.);
d) Aliquot II is incubated for 2 hours at 5° C. and pH 9.0;
e) Residual activity of aliquot II is measured (pH 9.0 and 25° C.);
f) Percentage residual activity of aliquot I relative to residual activity of aliquot II is calculated.

Alternatively, in the above definition of acid stability, the step b) buffer pH-value may be 1.0, 1.5, 2.0, 2.5, 3.0, 3.1, 3.2, 3.3, or 3.4.

In other alternative embodiments of the above acid stability definition relating to the above alternative step b) buffer pH-values, the residual protease activity as compared to the reference, is at least 5, 10, 15, 20, 25, 30, 35, 40, 45, 50, 55, 60, 65, 70, 75, 80, 85, 90, 95, or at least 97%.

In alternative embodiments, pH values of 6.0, 6.5, 7.0, 7.5, 8.0, or 8.5 can be applied for the step d) buffer.

In the above acid-stability definition, the term A280 = 1.0 means such concentration (dilution) of said pure protease which gives rise to an absorption of 1.0 at 280 nm in a 1 cm path length cuvette relative to a buffer blank.

And in the above acid-stability definition, the term pure protease refers to a sample with a A280/A260 ratio above or equal to 1.70.

Examples of proteases according to the invention are
a) a proteases derived from Nocardiopsis sp. NRRL 18262; or
b) proteases having at least 60%, at least 65%, at least 70%, at least 75%, at least 80%, at least 85%, at least 90%, or at least 95%, at least 96%, at least 97%, at least 98%, at least 99% or 100% sequence identity to any of the proteases of (a).
(c) proteases having at least 75%, such as at least 80%, such as at least 85%, preferably at least 90%, such as at least 95%, such as at least 96%, such as at least 97%, such as at least 98%, such as at least 99%, such as 100% sequence identity to SEQ ID NO:1, SEQ ID NO:2, or SEQ ID NO:3.
(d) proteases activity having an amino acid sequence having at least 80% sequence identity to SEQ ID NO:1; an amino acid sequence having at least 80% sequence identity to SEQ ID NO:2; or an amino acid sequence having at least 80% sequence identity to SEQ ID NO:3

In another particular embodiment, the protease according to the invention is thermostable.

Ronozyme ProAct is a preparation of serine protease produced by a genetically modified strain of Bacillus licheniformis. It is produced by fermentation of a sporulation deficient Bacillus licheniformis strain Rh 3 which expresses a synthetic gene encoding a serine protease (EC 3.4.21.-). Accordingly, in one aspect of the invention, the polypeptide having protease activity is produced by a genetically modified strain of Bacillus licheniformis, preferably a sporulation deficient Bacillus licheniformis strain Rh 3, and has least 70% sequence identity to a polypeptide having SEQ ID NO.1.

The polypeptide having protease activity typically has minimum protease activity levels of at least 35.000 PROT/kg, such as at least 50.000 PROT/kg, such as at least 75.000 PROT/kg. Ronozyme ProAct has protease activity of min. 75,000 prot / g. In relation to the polypeptide having protease activity, thermostability is intended to mean that the protease in the extrusion product maintains at least 60% of the 75.000 prot/g activity, such as at least 65% of the 75.000 prot/g activity, such as at least 70% of the 75.000 prot/g activity, such as at least 75% of the 75.000 prot/g activity, such as at least 80% of the 75.000 prot/g activity, such as at least 85% of the 75.000 prot/g activity, such as at least 90% of the 75.000 prot/g activity, such as at least 95% of the 75.000 prot/g activity of the polypeptide having protease activity of SEQ ID NO:1.

The term thermostable means one or more of the following: That the temperature optimum is at least 50° C., 52° C., 54° C., 56° C., 58° C., 60° C., 62° C., 64° C., 66° C., 68° C., or at least 70° C.

In a preferred embodiment, the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1;
(b) a variant of the polypeptide of SEQ ID NO: 1 comprising a substitution, deletion, and/or insertion of one or more (e.g. several) positions; and
(c) a fragment of the polypeptide of (a), or (b) that has protease activity.

In a more preferred embodiment, the polypeptide comprises or consists of SEQ ID NO: 1.

In another preferred embodiment, the enzyme granule of the present invention comprises or consists of a protease, dextrin and water, preferably an acid-stable protease, dextrin and water.

### An Extruded Enzyme Granule

One aspect of the invention is directed to a granule comprising a polypeptide having protease activity and having at least 70% sequence identity to the polypeptide of SEQ ID NO: 1; said granule prepared by an extrusion process.

An aspect of the invention is directed to an animal feed additive comprising a granule prepared by an extrusion process. An aspect of the invention is directed to a granule prepared by an extrusion process. One embodiment of the invention is directed to a formulation comprising the polypeptide of the invention as an extruded granule or prepared by a method comprising an extrusion process, typically said process comprising extruding a combination comprising said polypeptide, a meltable hydrophobic substance, and a solid carrier.

One aspect of the invention is directed to an animal feed additive comprising extruded enzyme pellets wherein said enzyme is a polypeptide having protease activity and having at least 70% sequence identity to SEQ ID NO:1. According to an aspect of the invention, the method of the present invention comprises (a) combining a polypeptide having protease activity, a solid carrier, optionally water, and a meltable hydrophobic substance to provide a combined product; (b) optionally applying sufficient heat to the combined product to allow the hydrophobic substance to melt; (c) extruding the product of step (b); and (d) allowing the extruded product of step (c) to dry and cool or actively drying and cooling the extruded product of step (c) to provide the thermostable enzyme product, wherein the polypeptide having protease activity has at least 70% sequence identity to SEQ ID NO: 1, namely at least 75% sequence identity to the polypeptide of RONOZYME® ProAct. A further aspect of the invention is directed to a method of preparing an animal feed additive comprising a polypeptide having protease activity having at least 70% sequence identity to SEQ ID NO: 1, namely having at least 75% sequence identity to the polypeptide of RONOZYME® ProAct, comprising an extrusion process, said process comprising extruding a combination comprising said polypeptide, a meltable hydrophobic substance, and a solid carrier. Another embodiment, the present invention encompasses a method for preparing a thermostable enzyme product for use in the manufacture of animal feed comprising (a) combining an enzyme, a solid carrier and a meltable hydrophobic substance to provide a combined product; (b1) reducing the moisture content by applying heat to the combined product and (b2) melting the hydrophobic substance; and (c) cooling the combined product to provide the thermostable enzyme product, wherein the thermostable enzyme is the polypeptide having protease activity and having at least 70% sequence identity to SEQ ID NO: 1.

In still another embodiment, the present invention encompasses a method for preparing a thermostable enzyme product for use in the manufacture of animal feed comprising (a) combining an enzyme, a solid carrier, a meltable hydrophobic substance to provide a combined product and optionally additional water to form a suitable paste; (b) optionally applying sufficient heat to the combined product to allow the hydrophobic substance to melt; (c) extruding the product of step (b); and (d) drying and cooling the extruded product of step (c) to provide the thermostable enzyme product. In one aspect of this embodiment, the meltable hydrophobic substance is added in step (a) as solid flakes or as a pre-melted molten liquid. The skilled person will recognize that if the meltable hydrophobic substance is added as a pre-melted molten liquid, step (b) may not be necessary. The components referred to in step (a) may be combined in a single step or alternatively, in separate steps. For example, the enzyme may first be combined with the solid carrier and optionally water, optionally dried, and then the resulting enzyme/carrier combination combined with the meltable hydrophobic substance.

### The Meltable Hydrophobic Substance

The meltable hydrophobic substance is typically selected from an oil and wax, such as selected from the group consisting of hydrogenated castor oil, hydrogenated palm kernel oil, hydrogenated rapeseed oil, hydrogenated palm oil, a blend of hydrogenated and unhydrogenated vegetable oil, 12-hydroxystearic acid, microcrystalline wax such as Cerit HOT, and high-melting paraffin waxes such as Mekon White.

A meltable hydrophobic substance according to the present invention includes, but is not limited to, oils and waxes, for example hydrogenated vegetable oils such as castor oil (HCO), palm kernel oil (HPKO), palm oil (FHPO or Akoflake Palm 58 (AP)) or rapeseed oil (FHRO or Akoflake FSR (AFx, where x= F (flake) or M (melt))), a blend of hydrogenated and unhydrogenated vegetable oil (PB3), 12-hyroxystearic acid (12-HSA), microcrystalline wax such as Cerit HOT, and high-melting paraffin waxes such as Mekon White. This meltable hydrophobic substance can be a single component or derived from mixtures of products designed to produce a desired melting point. This will include combinations with water immiscible liquids or low melting point hydrophobic solids that produce a mixture with a reduced melting point. These include waxes, C26 and higher, paraffin waxes, cholesterol, fatty alcohols, such as cetyl alcohol, mono-, di- and triglycerides of animal and vegetable origin such as tallow, hydrogenated fat, hydrogenated castor oil, fat derivatives such as fatty acids, soaps, esters, hydrophobic starches such as ethyl cellulose, lecithin. The waxes may be of natural origin, meaning they may be animal, vegetable or mineral. Animal waxes include, without limitation, beeswax, lanolin, shellac wax and Chinese insect wax. Vegetable wax includes, without limitation, camauba, candelilla, bayberry and sugar cane waxes. Mineral waxes include, without limitation, fossil or earth waxes including ozokerite, ceresin and montan or petroleum waxes, including paraffin and microcrystalline waxes. Alternatively the waxes may be synthetic or mixtures of natural and synthetic waxes. For instance, these can include low molecular weight partially oxidized polyethylene, which can be preferentially co-melted with paraffin. The fatty derivatives may be either fatty acids, fatty acid amides, fatty alcohols and fatty esters or mixtures of these. The acid amide may be stearamide. Sterols or long chain sterol, esters may also be such as cholesterol or ergosterol. The skilled person will recognize that combinations of two or more of the above mentioned waxes and/or oils may be employed.

By “meltable” hydrophobic substance it is meant a hydrophobic substance which is solid at the typical ambient storage temperature of a feed product but melts at a temperature above this. In one embodiment, the melting temperatures will range from 20° C. to 100° C. The upper temperature is limited by the ability to melt the hydrophobic substance in the process and the stability of the enzyme at these elevated temperatures for the processing period. In one aspect of this embodiment, the hydrophobic substance has a melting point in the range 20° C. to 95° C. C. In another aspect of this embodiment, the hydrophobic substance has a melting point in the range 25° C. to 90° C. In yet another aspect of this embodiment, the hydrophobic substance has a melting point in the range 20° C. to 80° C., such as from 20° C. to 70° C., such as from 20° C. to 65° C., such as from 20° C. to 60° C.

HCO is a hydrogenated castor oil with a typical melting point range of 82-86° C. PB3 is a blend of hydrogenated and non-hydrogenated vegetable oils with a typical melting point range of 38-46° C. Akoflake Palm 58 or FHPO is a hydrogenated (fully hardened) palm oil with a typical melting point range of 58-60° C. HPKO is a hardened palm kernel oil with a typical melting point range of 41-44° C. Akoflake FSR or FHRO is a hydrogenated (fully hardened) rapeseed oil with a typical melting point range of 66-69° C. It will be recognized by the person skilled in the art that the actual melting point may vary depending on environmental or physical conditions under which the meltable hydrophobic substance is heated, or the source of the meltable hydrophobic substance.

In one embodiment, the enzyme containing product of the present invention may comprise any suitable quantity of a meltable hydrophobic substance that protects the enzyme and maintains bioavailability. In one aspect of this embodiment, the enzyme containing product comprises 1-30% by weight of a meltable hydrophobic substance. In another aspect of this embodiment, the enzyme containing product comprises 5-20% by weight of a meltable hydrophobic substance. In another aspect of this embodiment, the enzyme containing product comprises at least 5% or more by weight, for example 7.5%, 10%, 20%, or 30% of a meltable hydrophobic substance. Without being bound by theory, it is thought that the treatment of the enzyme with a meltable hydrophobic substance protects the enzyme product matrix from the effect of temperature and moisture during the pelleting process. A sufficient concentration of a meltable hydrophobic substance is added to the matrix to effect the treatment and secure enhanced retention of activity of the enzyme, regardless of the concentration of enzyme present in the matrix.

### The Solid Carrier

In typical embodiments, solid carriers that are suitable for use in the method of the present invention include, without limitation, plant sourced absorbents such as ground seed grains, for example, ground corn, ground wheat, wheat middlings, soybean meal, rice hulls, corn gluten feed, corn grits, distiller’s dried grains, a mineral sourced absorbent, for example silica, diatomaceous earth or clay. In a more typical embodiment the solid carrier is ground wheat or corn. In another typical embodiment, the solid carrier is wheat or corn flour.

In one embodiment, the solid carrier is an absorbent and/or adsorbent material, such as a plant-based absorbent or a mineral sourced absorbent.

### Further Components

The skilled person will recognize that the present invention can be applied to protect other thermal process-labile components of animal feed concentrates, such as but not limited to any of the following groups, individually or in combination: vitamins, such as vitamin A, B12, C, D, D3, E, riboflavin, niacin, choline, folic acid etc.; nucleic acids and nucleotides etc., such as guanine, thymidine, cytosine, adenine etc.; amino acids, such as glycine, lysine, threonine, tryptophan, arginine, tyrosine, methionine etc.; micro-organisms, such as Aspergillus niger, A. oryzae, Bacillus subtilis, B. licheniformis, Lactobacillus acidophilus, L. bulgaricus etc.; medications and vaccines, such as chlortetracycline, erythromycin, oxytetracycline etc.; and flavour enhancers, such as sugars, spices, essential oils, synthetic flavourings.

### The Extrusion Process

According to the present invention the pelleting process for the preparation of the animal feed is an extrusion process. Typical extrusion processes for manufacturing feed pellets are known to those skilled in the art. Extrusion or pelletized products are products wherein the feed mixture (mash feed) is pressed to pellets or under pressure is extruded through a small opening and cut into particles which are subsequently dried. Such particles usually have a predeterminable size because of the material in which the extrusion opening is made (usually a plate with bore holes) sets a limit on the allowable pressure drop over the extrusion opening. Also, very high extrusion pressures increase heat generation in the mash feed when using a small opening. (Michael S. Showell (editor); Powdered detergents; Surfactant Science Series; 1998; vol. 71; page 140-142; Marcel Dekker).

In a particular embodiment, the mash feed is led to an extruder to form pellets of variable length from the extrudate. The extrusion apparatus may be any screw-type extruder known in the art. In a particular embodiment, the extruder is a double screwed extruder, e.g., a Werner & Pfleiderer Type continua 37″ extruder. Extrusion parameters (e.g., capacity, screw speed, die diameter, drying temperatures, drying time, etc.) are dependent upon the particular extrusion process and/or extrusion apparatuses employed.

In an embodiment, the screw speed of the extruder is 1-1,000 RPM. In a more particular embodiment, the screw speed of the extruder is 100 RPM. In an even more particular embodiment, the screw speed of the extruder is 150 RPM. In yet an even more particular embodiment, the screw speed of the extruder is 200 RPM. In still an even more particular embodiment, the screw speed of the extruder is 250 RPM. In still yet an even more particular embodiment, the screw speed of the extruder is 300 RPM.

In an embodiment, the die diameter is 0.5 mm - 5.0 mm. In a more particular embodiment, the die diameter is 0.5 mm. In an even more particular embodiment, the die diameter is 1.0 mm. In yet an even more particular embodiment, the die diameter is 1.5 mm. In a most particular embodiment, the die diameter is 2.0 mm.

The pellets are placed then dried for a specified time e.g., at least 15 minutes, preferably 20 minutes, at temperatures of 60-100° C., preferably 90-100° C., more preferably 90° C., even more preferably 95° C., even still more preferably 100° C.

One aspect of the invention is direct to a method of preparing an animal feed additive comprising a polypeptide having protease activity having at least 70% sequence identity to SEQ ID NO: 1, namely having at least 75% sequence identity to the polypeptide of RONOZYME® ProAct, or to a polypeptide as defined herein, comprising an extrusion process, said process comprising extruding a combination comprising said polypeptide, a meltable hydrophobic substance, and a solid carrier.

In still another embodiment, the polypeptide having protease activity having at least 70% sequence identity to SEQ ID NO: 1, is substantially stable when subjected to an extrusion process having a pressure of 1 bar to 40 bar and are subjected to an extrusion process wherein the extrusion process temperatures are temperatures from 60° C. to 100° C.

According to an aspect of the invention, the method of the present invention comprises (a) combining a polypeptide having protease activity, a solid carrier, optionally water, and a meltable hydrophobic substance to provide a combined product; (b) optionally applying sufficient heat to the combined product to allow the hydrophobic substance to melt; (c) extruding the product of step (b); and (d) allowing the extruded product of step (c) to dry and cool or actively drying and cooling the extruded product of step (c) to provide the thermostable enzyme product, wherein the polypeptide having protease activity has at least 70% sequence identity to SEQ ID NO: 1, namely at least 75% sequence identity to the polypeptide of RONOZYME® ProAct.

Another embodiment, the present invention encompasses a method for preparing a thermostable enzyme product for use in the manufacture of animal feed comprising (a) combining an enzyme, a solid carrier and a meltable hydrophobic substance to provide a combined product; (b1) reducing the moisture content by applying heat to the combined product and (b2) melting the hydrophobic substance; and (c) cooling the combined product to provide the thermostable enzyme product, wherein the thermostable enzyme is the polypeptide having protease activity and having at least 70% sequence identity to SEQ ID NO: 1.

Another embodiment, the present invention encompasses a method for preparing a thermostable enzyme product for use in the manufacture of animal feed comprising (a) combining an enzyme, a solid carrier and a meltable hydrophobic substance to provide a combined product; (b1) reducing the moisture content by applying heat to the combined product and (b2) melting the hydrophobic substance; and (c) cooling the combined product to provide the thermostable enzyme product, wherein the thermostable enzyme is the polypeptide having protease activity and having at least 70% sequence identity to SEQ ID NO: 1.

In still another embodiment, the present invention encompasses a method for preparing a thermostable enzyme product for use in the manufacture of animal feed comprising (a) combining an enzyme, a solid carrier, a meltable hydrophobic substance to provide a combined product and optionally additional water to form a suitable paste; (b) optionally applying sufficient heat to the combined product to allow the hydrophobic substance to melt; (c) extruding the product of step (b); and (d) drying and cooling the extruded product of step (c) to provide the thermostable enzyme product. In one aspect of this embodiment, the meltable hydrophobic substance is added in step (a) as solid flakes or as a pre-melted molten liquid. The skilled person will recognize that if the meltable hydrophobic substance is added as a pre-melted molten liquid, step (b) may not be necessary. The components referred to in step (a) may be combined in a single step or alternatively, in separate steps. For example, the enzyme may first be combined with the solid carrier and optionally water, optionally dried, and then the resulting enzyme/carrier combination combined with the meltable hydrophobic substance.

In still another embodiment, the present invention encompasses a method for preparing a thermostable enzyme product for use in the manufacture of animal feed comprising (a) combining a polypeptide having protease activity and having at least 70% sequence identity to SEQ ID NO: 1, a solid carrier, a meltable hydrophobic substance to provide a combined product and optionally additional water to form a suitable paste; (b) melting the hydrophobic substance, or allowing the hydrophobic to melt, optionally by applying heat to the combined product; (c) extruding the product of step (b); and (d) optionally drying and cooling the extruded product of step (c) to provide the thermostable enzyme product.

In one aspect of this embodiment, the meltable hydrophobic substance is added in step (a) as solid flakes or as a pre-melted molten liquid. The skilled person will recognize that if the meltable hydrophobic substance is added as a pre-melted molten liquid, step (b) may not be necessary. The components referred to in step (a) may be combined in a single step or alternatively, in separate steps. For example, the enzyme may first be combined with the solid carrier and optionally water, optionally dried, and then the resulting enzyme/carrier combination combined with the meltable hydrophobic substance.

In a further embodiment of the invention, the meltable hydrophobic substance-treated enzyme product of the invention is mixed with suitable feed agents and compounded via a heating/pelleting process to produce an animal feed containing a prescribed amount of the protease enzyme. This process typically involves 1. mixing all the components together, namely the polypeptide having protease activity, the meltable hydrophobic substance, and the solid carrier; 2. compressing them though an extruder, optionally with steam injection to act as a binder, to produce suitable feed pellets for administration to animals (such as, but not limited to, poultry or swine).

During this process the temperatures of the feed (referred to as the “mash”) can be raised to about 90° C. At these temperatures, most enzymes may be deactivated rapidly. The product of this process is then assayed for recovery of enzyme (expressed as % recovered relative to the equivalent, non- processed mash used to prepare the pellets). The product of an original granulation process serves as a comparison.

In a further embodiment, the solid and liquid ingredients of the feed are premixed except for a liquid binder ingredient which is mixed in last. The resulting mash is extruded in a ring die pellet extruder with or without steam conditioning, preferably without and the extruded pellets are cooled and/or dried as may be required. The liquid binder will have viscous and cohesive properties and preferably will be a condensed liquid byproduct from the grain, food or feed processing industries.

As discussed, it has been surprisingly found that the polypeptide of SEQ ID NO:1 may be formulated as an extrudate, wherein the extruding process may comprise the use of elevated temperatures without substantial loss in activity, including the use of steam. However, the process may alternatively eliminate the conditioning step involving the use of steam and/or elevated temperatures and instead involve a “cold” pelleting process. In the cold pelleting process of the present invention, liquid binders are used in place of steam. The binders are animal feed ingredients in themselves and have viscous and cohesive properties. When the liquid binder is applied to the other feed ingredients, free moisture penetrates solid particles in the meal while the viscous cohesive substances in the binder agglomerate fine particles into larger particles and then remain on the surfaces of the large solid particles, creating a cohesive surface. When the resulting moist cohesive mash is compressed through the die, the particles are compacted and bound together to form pellets having enhanced durability.

In the cold pelleting extrusion process, after batching, the dry ingredients are mixed in the mixer. Then the liquid ingredients, such as fat or molasses, are added and mixed. Liquid binder is typically added last by blending the binder into the mix to obtain a uniform cohesive mash. Liquid binders may be used at a rate of 5 to 25% by weight in a formula, with 10 to 20% being preferred for cold pelleting. Liquid feed ingredients are usually relatively economical nutrient sources being condensed liquid by-products from the grain, food or feed processing industries, such as molasses and fat. Liquid binder may be used in conventional extrusion processes involve heat or stem. However, the amount of those liquids is usually restricted to less than 6% in a conventional pelleting process.

In the conventional pelleting processes, meal conditioning with steam is a prerequisite for the compression of the meal or mash into pellets. Heat and water from the steam serve to activate binders in the meal particles (i.e. protein and carbohydrates), soften them and bring cohesive properties onto the surfaces of the particles. When the mash is compressed through a die, the particles are compacted and stuck together to form pellets. In the cold pelleting process of the present invention, liquid binders are used instead of steam. The binders have viscous and cohesive properties. When such a liquid binder is applied, free moisture penetrates solid particles in the mash while the viscous, cohesive substances in the binder agglomerate fine particles into larger particles and then remain on the surfaces of large solid particles, creating cohesive surfaces. When the moist, cohesive mash is compressed through a die, the particles are compacted and bound together to form durable pellets. Liquid binders used in the cold pelleting process can be any condensed liquid byproducts from the grain, food or feed processing industries. The liquid binders should have a solids content of 20-80% by weight, preferably 35-65%, and should have viscous and cohesive properties. Typical liquid binders include Brewex (a concentrated molasses-like by-product of the brewing industry), corn steep liquor, condensed porcine solubles, condensed distillery solubles, molasses, desugared molasses, sugar syrup, and condensed liquid whey.

In the cold pelleting process the pellets discharge from the pellet extruder die at a temperature of 35 to 70° C., typically 37 to 65° C., usually below 55° C., depending upon the diet formula, type of liquid binders and levels of binder used. In contrast, in conventional pelleting processes the pellets may have temperatures of 60 to 100° C. The low temperatures of the pellets of the present invention provide an opportunity to incorporate heat sensitive and labile substances and feed ingredients such as other enzymes than SEQ ID NO:1, microbials, and milk proteins or other feed ingredients which can be destroyed and/or rendered nutritionally unavailable by heat in conventional pelleting processes.

A further aspect of the invention is directed to a method of producing animal feed pellets by an extrusion process described herein.

The present invention further provides a product obtainable by a method of the invention, a method for preparing an animal feed comprising combining a product obtainable by a method of the present invention with suitable animal feed ingredients and an animal feed so produced.

As can be seen from Example 10 and Example 12, a granule prepared by an extrusion process according to the invention has a high activity after the being subjected to pelleting model thermostability studies, higher than many commercial products in animal feed.

### A Granule Prepared by a Spray-Drying Process

### The Granule

One aspect of the invention is directed to a granule comprising a polypeptide having protease activity and having at least 70% sequence identity to the polypeptide of SEQ ID NO: 1; said granule prepared by a spray-drying process.

The granule according typically further comprises a carbohydrate. The carbohydrate is preferably selected from the group consisting of lactose, sucrose, mannitol, α-cyclodextrin and dextrin, more preferably dextrin.

The granule according to this aspect, the granule typically comprises a protease selected from the group consisting of:
(a) a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1;
(b) a variant of the polypeptide of SEQ ID NO: 1, comprising a substitution, deletion, and/or insertion of one or more (e.g. several) positions; and
(c) a fragment of the polypeptide of (a) or (b) that has protease activity.
In the granule according to this aspect of the invention, the polypeptide may comprise or consist of SEQ ID NO: 1.

The granule may be produced by a spray drying process comprising (a) preparing a spray liquid comprising a polypeptide having protease activity and having at least 70% sequence identity to the polypeptide of SEQ ID NO: 1; and a carbohydrate. The granule suitably comprises water and typically has a water content of less than 7%. The granule therefore typically comprises or consists of an acid-stable protease, dextrin and water.

The enzyme granule prepared by a spray-drying process of the invention has a simple structure, comprising a protease and a suitably a carbohydrate, such as dextrin. The enzyme granule prepared by a spray-drying process has an excellent enzyme performance, including pH-stability and temperature-activity, while reducing the cost of granulation and coating (both process costs and raw material costs. In a preferred embodiment of a granule prepared by a spray-drying process, the residual activity of the enzyme granule of the invention is maintained by at least 45, 50, 55, 60, 65, 70, 75, 80, 85, 90, 95, or at least 97% of the reference activity after at least 5 days, 30 days, 2 months or 1 year of storage at an ambient temperature. In a more preferred embodiment, the residual activity of the enzyme granule of the invention is maintained by at least 65, 70, 75, 80, 85, 90, 95, or at least 97% of the reference activity after at least 5 days, 30 days, 2 months or 1 year of storage at an ambient temperature. In a preferred embodiment, the acid-stability of the enzyme granule of the invention is at least 45, 50, 55, 60, 65, 70, 75, 80, 85, 90, 95, or at least 97% of the reference activity. In a more preferred embodiment, the acid-stability of the enzyme granule of the invention is at least 65, 70, 75, 80, 85, 90, 95, or at least 97% of the reference activity. In a preferred embodiment, the temperature activity of the enzyme granule of the invention is at least 45, 50, 55, 60, 65, 70, 75, 80, 85, 90, 95, or at least 97% of the reference activity. In a more preferred embodiment, the temperature activity of the enzyme granule of the invention is at least 65, 70, 75, 80, 85, 90, 95, or at least 97% of the reference activity.

### Preparation of the Granule Prepared by a Spray-Drying Process

In one aspect, the present invention relates a method of producing an enzyme granule, comprising
(a) preparing a spray liquid comprising an acid-stable protease and a carbohydrate; and
(b) spraying the spray liquid in a spray tower.

In one embodiment, the method of producing an enzyme granule, comprises
(a) preparing a spray liquid comprising a polypeptide having protease activity and having at least 70% sequence identity to the polypeptide of SEQ ID NO: 1; and a carbohydrate; and
(b) spraying the spray liquid in a spray tower.

In the method of preparing a granule by a spray-drying process, the carbohydrate may be selected from the group consisting of lactose, sucrose, mannitol, α-cyclodextrin and dextrin, preferably dextrin. Typically, the spray tower has an inlet temperature of 100-200° C. and/or a product temperature of 50-80° C.

Methods for preparing the enzyme granule prepared by a spray-drying process can be found in Handbook of Powder Technology; Particle size enlargement by C. E. Capes; Volume 1; 1980; Elsevier. In a preferred embodiment, the enzyme granule is produced by spray drying. The spray is typically the carbohydrate is selected from the group consisting of lactose, sucrose, mannitol, α-cyclodextrin and dextrin. The dextrin is typically a white dextrin.

Spray dried products, wherein a liquid enzyme-containing solution is atomized in a spray drying tower to form small droplets which during their way down the drying tower dry to form a continuous film layer which encapsulate the enzyme-containing particles. Very small particles can be produced this way (Michael S. Showell (editor); Powdered detergents; Surfactant Science Series; 1998; vol. 71; page 140-142; Marcel Dekker).

After drying, the enzyme granules preferably contain 0.1-10 % w/w water, preferably 1, 2, 3, 4, 5, 6 or 7% w/w water.

An aspect of the invention is directed to an enzyme granule for use in animal feed, said granule defined prepared by a spray-drying process. A further aspect is directed animal feed comprising the granule prepared by a spray-drying process. A related aspect is directed to use of the enzyme granule prepared by a spray-drying process in an animal feed.

As can be seen from Example 11, a granule prepared by a spray-drying process process according to the invention has a high activity after the being subjected to pelleting model thermostability studies.

### A Granule Comprising a Salt Core and Protease-Containing Layer (a Microgranule)

### The Granule (the Microgranule)

The enzyme granule comprising a salt core and a protease-containing layer, typically comprises a sodium sulfate or sodium chloride core, and a protease containing layer. The protease in the protease-containing layer is typically an acid-stable protease. The enzyme granule comprising a salt core and an protease-containing layer has an excellent enzyme performance, including pH-stability and temperature-activity, while reducing the cost of granulation and coating (both process costs and raw material costs). In a preferred embodiment, the residual activity of the enzyme granule comprising a salt core and an acid-stable protease containing layer is maintained by at least 45, 50, 55, 60, 65, 70, 75, 80, 85, 90, 95, or at least 97% of the reference activity after at least 5 days, 30 days, 2 months or 1 year of storage at an ambient temperature. In a more preferred embodiment, the residual activity of the enzyme granule comprising a salt core and an acid-stable protease containing layer is maintained by at least 65, 70, 75, 80, 85, 90, 95, or at least 97% of the reference activity after at least 5 days, 30 days, 2 months or 1 year of storage at an ambient temperature. In a preferred embodiment, the acid-stability of the enzyme granule comprising a salt core and an acid-stable protease containing layer is at least 45, 50, 55, 60, 65, 70, 75, 80, 85, 90, 95, or at least 97% of the reference activity. In a more preferred embodiment, the acid-stability of the enzyme granule comprising a salt core and an acid-stable protease containing layer is at least 65, 70, 75, 80, 85, 90, 95, or at least 97% of the reference activity. In a preferred embodiment, the temperature activity of the enzyme granule comprising a salt core and an acid-stable protease containing layer is at least 45, 50, 55, 60, 65, 70, 75, 80, 85, 90, 95, or at least 97% of the reference activity. In a more preferred embodiment, the temperature activity of the enzyme granule of the invention is at least 65, 70, 75, 80, 85, 90, 95, or at least 97% of the reference activity.

### Preparation of the Granule Comprising a Salt Core and a Protease-Containing Layer

In one aspect, the present invention relates to an enzyme granule comprising a salt core and a protease containing layer, preferably comprising a sodium sulfate or sodium chloride core, and a protease containing layer. Methods for preparing the enzyme granule can be found in Handbook of Powder Technology; Particle size enlargement by C. E. Capes; Volume 1; 1980; Elsevier.

In a preferred embodiment, the enzyme granule comprising a salt core and a protease containing layer is prepared by fluid bed granulation.

i) Fluid bed granulation involves suspending particulates in an air stream and spraying a liquid onto the fluidized particles via nozzles. Particles hit by spray droplets get wetted and become tacky.

ii) The cores may be subjected to drying, such as in a fluid bed drier. Other known methods for drying granules in the feed or enzyme industry can be used by the skilled person. The drying preferably takes place at a product temperature of from 25 to 90° C. For some enzymes it is important the enzyme granules contain a low amount of water before coating with the salt. If water sensitive enzymes are coated with a salt before excessive water is removed, it will affect the activity of the enzyme negatively. After drying, the cores preferably contain 0.1-10 % w/w water, preferably 1, 2, 3, 4, or 5% w/w water.

### A Sodium Sulfate or Sodium Chloride Core

The core may comprise a single salt or a mixture of two or more salts. The salt may be water soluble, in particular having a solubility at least 0.1 grams in 100 g of water at 20° C., preferably at least 0.5 g per 100 g water, e.g. at least 1 g per 100 g water, e.g. at least 5 g per 100 g water.

The salt may be an inorganic salt, e.g. salts of sulfate. The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous sodium chloride (NaCl). In a preferred embodiment, the salt is selected from the group consisting of sodium sulfate and sodium chloride.

### The Protease Containing Layer

Preferably the acid stable protease is applied to the salt core as a granulation fluid or as a liquid (for example, protease concentrate, dissolved in buffer or water) e.g. using a fluid bed, as known in the art.

The protease is typically selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1;
(b) a variant of the polypeptide of SEQ ID NO: 1, comprising a substitution, deletion, and/or insertion of one or more (e.g. several) positions; and
(c) a fragment of the polypeptide of (a) or (b) that has protease activity.

The protease is selected from a polypeptide having at least 75%, such as at least 80%, such as at least 85%, preferably at least 90%, such as at least 95%, such as at least 96%, such as at least 97%, such as at least 98%, such as at least 99%, such as 100% sequence identity to SEQ ID NO:1, SEQ ID NO:2, or SEQ ID NO:3. The enzyme granule typically comprises SEQ ID NO: 1, SEQ ID NO: 2 or SEQ OD NO:3.

### Optional Additional Coating

The granule may optionally have one or more additional coatings. Examples of suitable coating materials are polyethylene glycol (PEG), methyl hydroxy-propyl cellulose (MHPC) and polyvinyl alcohol (PVA).

The enzyme granule is typically a microgranule, having a particle size of 100-2000 micrometers, preferably 200-1500 micrometers, more preferably 300-1200 micrometers, the granule has a water content of less than 5%.

The method of producing an microgranule, suitably comprises
(a) preparing a sodium sulfate, or sodium chloride core; and
(b) distributing a protease liquid to the sodium sulfate, or sodium chloride core.

The protease liquid may be distributed onto the sodium sulfate or sodium chloride core by spray. Typically, the granule is prepared in a fluid bed apparatus.

### A High-Shear Granulate

An aspect of the invention is directed to an animal feed additive comprising a polypeptide having protease activity, namely a polypeptide having at least 70% sequence identity to SEQ ID NO:1 in a granule or granulate prepared by a high-shear granulation process.

A high-shear granulation process allows for a pelleting-stable granulate of a polypeptide having protease activity, namely a polypeptide having at least 70% sequence identity to SEQ ID NO:1. A polypeptide having at least 70% sequence identity with a polypeptide of SEQ ID NO:1 is known to be an excellent zootechnical additive to animal feed. An aspect of the invention is directed animal feed additive comprising a polypeptide having protease activity and having at least 70% sequence identity to SEQ ID NO:1 said polypeptide in granule or granulate prepared by a high-shear granulation process.

### The Granulate

An aspect of the invention is directed to an enzyme granulate said granulate prepared by a method comprising a high-shear granulation process, said granulate comprising a polypeptide having protease activity, said polypeptide having at least 70% sequence identity with a polypeptide of SEQ ID NO:1.

The high-shear granulation process typically comprises
A. forming a powder mixture by combining at least
   i. cellulose or a derivative thereof
   ii. a binder; and
   iii. optionally a filler; and
B. adding a liquid phase granulating agent
wherein the polypeptide having protease activity is added to either the powder mixture or to the liquid phase granulating agent.

The enzyme granulate, prepared by a high-shear granulation process, typically has a density from 0.35 to 0.8, such as 0.37 to 0.7, such as 0.40 to 0.6.

### The Cellulose

The enzyme granulate comprises cellulose or a derivative thereof. Many commercial cellulose sources are suitable and known to the person skilled in the art. Typically, the cellulose or a derivative thereof is in fibrous form or is a microcrystalline cellulose.

Examples of suitable cellulose include the cellulose powder-CEPO S 20 (The Swedish cellulose powder and Wood Flour Mills Ltd.) and the cellulose Arbocel BC200.

Several brands of cellulose in fibrous form are on the market, e.g. CEPO and ARBOCEL. In a publication from Svenska Tramjolsfabrikerna AB, “Cepo Cellulose Powder” it is stated that for Cepo S/20 cellulose the approximate miximum fibre length is 500 mu, the approximate average fibre length is 160 mu, the approximate maximum fibre width is 50 mu and the approximate average fibre width is 30 mu. Also it is stated that CEPO SS/200 cellulose has an approximate maximum fibre length of 150 mu, an approximate average fibre length of 50 mu an approximate maximum fibre width of 45 mu and an approximate average fibre width of 25 mu. Cellulose fibres with these dimensions are very well suited for the purpose of the invention.

The cellulose in fibrous form can be sawdust, pure, fibrous cellulose, cotton, or other forms of pure or impure fibrous cellulose.

The cellulose and cellulose derivatives may be selected from the group consisting of hydroxypropyl cellulose, methyl cellulose or carboxymethyl cellulose (CMC).

A preferred embodiment of the process according to the invention comprises the use of between 5 and 30 percent by weight of cellulose or cellulose derivative.

### The Binder

The enzyme granulate comprises a binder. The binder is typically selected from the group consisting of polyvinyl pyrrolidone, titanium dioxide, dextrins, polyvinylalcohol, polyethylene glycol, cellulose and cellulose derivatives, such as hydroxypropyl cellulose, methyl cellulose or carboxymethyl cellulose (CMC), such as polyvinyl pyrrolidone, titanium dioxide, dextrins, polyvinylalcohol, cellulose and cellulose derivatives

### Dextrin W80 Is a Suitable Dextrin.

### The Filler

The filler may be any component which does not interfere with the granulating process, such as inorganic salts. This may include any salt comprising a one or more anions selected from the group consisting of CO₃^{2—} , SO₄^{2—}, HPO₄^{2—} ,H₂PO₄^{—} , F^{—}, Cl^{—}, Br^{—} , NO₃^{—} , I^{—} ,ClO₄^{—} , and SCN⁻ for anions, and cations selected from the group consisting of Na⁺ > K⁺ > Mg²⁺ > Ca²⁺. A typically embodiment is selected from the group consisting of NaCl, CaCO₃, Na₂SO₄, CaCl, and NaHCO₃, typically NaCl, CaCO₃, Na₂SO₄.

### The Granulating Agent

Using high shear granulation, an enzyme granulate can be produced without unwanted layer of starting material for the granulation on the walls of the drum granulator. With high shear granulation, the powder mixture being granulated is less sensitive to the granulating agent.

More specifically, the process for the production of enzyme granulates according to the present invention suitably comprises the introduction into the drum granulator of from 2 to 40 percent by weight of cellulose in fibrous form, from 0 to 10 percent by weight of a binder as herein defined, enzyme and filler in an amount which generates the intended enzyme activity in the finished granulate, a liquid phase granulating agent consisting of a waxy substance, as defined herein, and/or water, in an amount of between 5 and 70 percent by weight, whereby the maximum amount of waxy substance is 40 percent by weight and the maximum amount of water is 70 percent by weight, whereby all percentages are referring to the total amount of dry substances, the sequence of the introduction of the different materials being arbitrary, except that at least a major part of the granulating agent is introduced after at least a substantial part of the dry substances is introduced in the granulator, whereafter the granulate if necessary is dried in a conventional manner, preferably in a fluid bed.

The binders used in the process according to the invention are the binders conventionally used in the field of granulation with a high melting point or with no melting point at all and of a non waxy nature, e.g. polyvinyl pyrrolidone, dextrins, polyvinylalcohol, and cellulose derivatives, including for example hydroxypropyl cellulose, methyl cellulose or CMC. A granulate can not be formed on the basis of cellulose, enzyme, filler and a binder, as above defined, without the use of a granulating agent.

The filler is typically used for the purpose of adjusting to the intended enzyme activity in the finished granulate. Since the enzyme introduced into the granulator already contains diluents which are considered as fillers, additional filler is not always needed to standardize the enzymatic activity of the granulate. If a filler is used, it may typically be NaCl, but other components acting as fillers which do not interfere with the granulating process and later use of the product can be used, especially other inorganic salts.

The liquid phase granulating agent may be selected from the group consisting of a waxy substance and/or water or aqueous solution. The granulating agent may be water and/or a waxy substance. The granulating agent is always used as a liquid phase in the granulation process; the waxy substance if present therefore is either dissolved or dispersed in the water or melted. By a waxy substance is understood a substance which has a melting point is between 30° C. and 100° C., preferably between 40° C. and 60° C.

Both water and waxy substance are granulating agents, i.e. they are both active during the formation of the granules; the waxy substance stays as a constituent in the finished granules, whereas the majority of the water is removed during the drying. Thus, in order to refer all amounts to the finished, dry granules all percentages are calculated on the basis of total dry substances, which means that water, one of the granulating agents, is not added to the other constituents when calculating the percentage of water, whereas the waxy substance, the other granulating agent, has to be added to the other dry constituents when calculating the percentage of waxy substance. Examples of waxy substances are polyglycols, fatty alcohols, ethoxylated fatty alcohols, higher fatty acids, mono-, di- and triglycerolesters of higher fatty acids, e.g. glycerol monostearate, alkylarylethoxylates, and coconut monoethanolamide.

If a high amount of waxy substance is used, relatively little or no water is added, and vice versa. Thus the granulating agent can be either water alone, waxy substance alone or a mixture of water and waxy substance. In case a mixture of water and waxy substance is used, the water and the waxy substance can be added in any sequence, e.g. first the water and then the waxy substance, or first the waxy substance and then the water or a solution or suspension of the waxy substance in the water. Also, in case a mixture of water and waxy substance is used, the waxy substance can be soluble or insoluble (but dispersable) in water.

If no water is used in the granulating agent, usually no drying is needed. In this case the granulating agent is a melted waxy material, and only cooling is needed to solidify the particles. In most cases, however, some drying is performed, and the drying is usually carried out as a fluid bed drying whereby small amounts of dust and small granules are blown away from the surface of the granules. However, any kind of drying can be used. In the instance where no water is used as a granulating agent, a flow conditioner or anticaking agent may be added to the granulate either before or after the cooling step, e.g. fumed silica, for instance the commercial products AEROSIL or CAB-OSIL.

A further aspect of the invention is directed to a method of preparing a granulate comprising a granulate comprising
a said high-shear granulation process comprising
   A. forming a powder mixture by combining at least
      i. cellulose or a derivative thereof
      ii. optionally a binder; and
      iii. optionally a filler; and
   B. adding a liquid phase granulating agent
   wherein the polypeptide having protease activity is added to either the powder mixture or to the liquid phase granulating agent and wherein the at least one binder is added to either the powder mixture or to the liquid phase granulating agent or both;
wherein said polypeptide having protease activity is a polypeptide having at least 70% sequence identity to SEQ ID NO:1,
or wherein said high-shear granulation process comprises
   A′. forming a powder mixture by combining at least
      i. cellulose or a derivative thereof
      ii. a binder; and
      iii. optionally a filler; and
   B′. adding a liquid phase granulating agent
   wherein the polypeptide having protease activity is added to either the powder mixture or to the liquid phase granulating agent,
wherein said polypeptide having protease activity is a polypeptide having at least 70% sequence identity to SEQ ID NO:1.

Typically, the polypeptide has at least 70% sequence identity with a polypeptide of SEQ ID NO:1 and has protease activity, such as at least 75% sequence identity with a polypeptide of SEQ ID NO:1, such as at least 80% sequence identity with a polypeptide of SEQ ID NO:1,such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100%.

In a further typical embodiment, the polypeptide having protease activity comprises a polypeptide sequence having at least 70% sequence identity with a polypeptide of SEQ ID NO:1 and further comprises an N-terminal sequence 1 to 30 amino acid residues and/or a C-terminal sequence of 1 to 30 amino acid residues. The polypeptide having protease activity may comprises a polypeptide sequence having at least 75% sequence identity with a polypeptide of SEQ ID NO:1 such as at least 75% sequence identity with a polypeptide of SEQ ID NO:1, such as at least 80% sequence identity with a polypeptide of SEQ ID NO:1, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100%, and further comprises an N-terminal sequence 1 to 30 amino acid residues and/or a C-terminal sequence of 1 to 30 amino acid residues.

The protease, after high-sheer granulation, typically has minimum enzyme activity levels of 15.000 PROT/kg.

The granulator can be any of the known types of mixing granulators, drum granulators, pan granulators or modifications of these. If a mixing granulator is used, for example a mixing drum from the German Company Gebr. Lodige Maschinen G.m.b.H, 479 Paderborn, Elsenerstrasse 7-9, DT, it is preferred that small rotating knives are mounted in the granulator in order to compact the granules.

A preferred embodiment of the process according to the invention comprises a granulation carried out at 50-70° C.

The enzyme granulate produced by the high-shear granulation process typically provides dry granulates have a diameter between 0.2 to 2 mm, such as 0.3 to 1.5 mm.

Preferably, all the solid materials are added first to the granulator, whereafter a homogeneous mixture is created and then the granulating agent is introduced as a spray (from one or more of the nozzles present on the granulator).

Usually, the filling volume of the total solid starting materials is below 50 percent of the total volume of the granulator, preferably below 30 percent of the total volume of the granulator.

With the granulation according to practice of the invention it is possible to avoid excessive recirculation of granules which are too fine and to large; actually only about 20 percent of the granules are recirculated as an average.

The high-sheer granulation process typically comprises.
1. The composition of a given composition as a dry powder.
2. Mixing of the dry powder composition.
3. Wetting of the powder mixture with granulating agent e.g. water or a water/binder solution.
4. Processing of the wet powder mixture with the granulating apparatus (rotating knife) until the granulate has the desired particle distribution and degree of roundness.
5. Fluid bed drying of the moist granulate until a dryness which satisfies both the requirements of enzyme stability and the requirements of free-flowing properties and mechanical strength.

Usually this will correspond to a water content less than 10 percent, preferably less than 3 percent.

### Animal Feed and Animal Feed Additive

The present invention is also directed to methods for the granules of the invention in preparation of an enzyme-enriched animal feed, as well as to animal feed and feed additives comprising the granules of the invention.

In particular embodiments, the granules of the invention are for use in feed for (i) non-ruminant animals; preferably (ii) mono-gastric animals; more preferably (iii) pigs, poultry, fish, and crustaceans; or, most preferably, (iv) pigs and poultry.

The granules of the invention can be fed to the animal before, after, or simultaneously with the diet. The latter is preferred.

The term feed, feed composition, or diet means any compound, preparation, mixture, or composition suitable for, or intended for intake by an animal. More information about animal feed compositions is found below.

In one embodiment, the present invention relates to an animal feed comprising a granule of the invention. The granules of the invention provide additional protein digestibility on top of endogenous proteases, resulting in a 3-6 % increase in amino acid digestibility. The granules of the invention increase energy (ME) by at least 25 kcal/kg diet.

The granules contribute to sustainable poultry production by supporting:
1. efficient use of natural resources: lower soy utilization and more alternative (local and by-products) raw materials
2. reduction of livestock emissions: low Nitrogen emissions by enabling lower Crude Protein diets
3. lifetime performance & animal welfare: significantly reducing foot-pad lesions Specifically developed for inclusion in animal diets, SEQ ID NO:1 significantly increases the protein digestion. It complements naturally occurring proteases, and considerably increases peptide supply as so to enhance animal performance. It improves the digestibility of a wide range of protein sources and cereals, allowing savings in feed costs.

In a preferred embodiment of the invention, the animal feed comprises 100 to 500 g protease/mT of feed, such as 100 to 300 g/mT, such as 125 to 250 g /mT. In a preferred embodiment for broiler chickens, the animal feed comprises the granules so as to comprise 150 to 250 g protease/ mT of feed, such as 175 g/mT to 225 g/mT, such as 200 g/mT for broiler chickens.

In a preferred embodiment for broiler chickens, the animal feed comprises the granules so as to comprise 100 to 200 g protease/ mT of feed, such as 125 g/mT to 175 g/mT, such as 150 g/mT for layers & breeders

In a further aspect, the present invention relates to an animal feed additive, comprising a granule of the invention and one or more additional components selected from the group consisting of: one or more vitamins; one or more minerals; one or more amino acids; one or more phytogenics; one or more prebiotics; one or more organic acids; and one or more other feed ingredients. The following are non-exclusive lists of examples of these components:

Examples of fat-soluble vitamins are vitamin A, vitamin D3, vitamin E, and vitamin K, e.g. vitamin K3.

Examples of water-soluble vitamins are vitamin B12, biotin and choline, vitamin B1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

Examples of amino acids which are used in animal feed are lysine, alanine, beta-alanine, threonine, methionine and tryptophan.

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products). Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are propionic acid, formic acid, citric acid, lactic acid, sorbic acid, malic acid, acetic acid, fumaric acid, benzoic acid, butyric acid and tartaric acid or their salt (typically sodium or potassium salt such as potassium diformate or sodium butyrate).

Further, optional, feed-additive ingredients are colouring agents, e.g. carotenoids such as beta-carotene, astaxanthin, and lutein; aroma compounds; stabilisers; antimicrobial peptides; polyunsaturated fatty acids; reactive oxygen generating species; and/or at least one other enzyme selected from amongst another pectinase (EC 3.2.1.8); and/or beta-glucanase (EC 3.2.1.4 or EC 3.2.1.6).

Examples of antimicrobial peptides (AMP’s) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP’s) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Usually fat- and water-soluble vitamins, as well as trace minerals form part of a so-called premix intended for addition to the feed, whereas macro minerals are usually separately added to the feed. A premix enriched with a granule of the invention is an example of an animal feed additive of the invention.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

WO 01/58275 corresponds to U.S. Pat. No. 6,960,462 which is hereby incorporated by reference.

An animal feed composition according to the invention has a crude protein content of 50-800 g/kg (preferably 50-600 g/kg, more preferably 60-500 g/kg, even more preferably 70-500, and most preferably 80-400 g/kg) and furthermore comprises at least one fiber-degrading enzyme as claimed herein. In additional preferred embodiments, the crude protein content is 150-800, 160-800, 170-800, 180-800, 190-800, or 200-800 - all in g/kg (dry matter). In particular embodiments, the crude protein content comes from oil seed material of the present invention.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg) = N (g/kg) x 6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

In a further aspect, the present invention relates to a method of improving the Average Metabolizable Energy of plant-based diet in a monogastric animal comprising administering an animal feed additive of the present invention or the animal feed of the present invention.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

### Preferred Embodiments

1. An animal feed additive comprising a polypeptide having protease activity, wherein the protease comprises a polypeptide having at least 70% sequence identity to SEQ ID NO:1; characterized in that the enzyme is formulated in a formulation selected from the group consisting of:
i. a granule prepared by an extrusion process;
ii. a granule prepared by a spray-drying process;
iii. a granule comprising a salt core, such as a sodium sulfate or sodium chloride core, and a protease-containing layer; and
iv. a granule prepared by a high-shear granulation process

2. The animal feed additive according to paragraph 1, wherein the polypeptide having protease activity is obtained or obtainable from Nocardiopsis sp. NRRL 18262.

3. The animal feed additive according to paragraph 1 or 2, wherein the polypeptide having protease activity has at least 75% sequence identity with a polypeptide of SEQ ID NO:1, such as at least 80% sequence identity with a polypeptide of SEQ ID NO:1, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100%.

4. The animal feed additive according to paragraphs 1 to 3, wherein the polypeptide having protease activity is selected from a polypeptide having at least 75%, such as at least 80%, such as at least 85%, preferably at least 90%, such as at least 95%, such as at least 96%, such as at least 97%, such as at least 98%, such as at least 99%, such as 100% sequence identity to SEQ ID NO:1, SEQ ID NO:2, or SEQ ID NO:3.

5. The animal feed additive according to paragraphs 1 to 4, wherein the polypeptide having protease activity is selected from the group consisting of
i. a polypeptide having at least 80% sequence identity to SEQ ID NO:1, having protease activity;
ii. a polypeptide having at least 80% sequence identity to SEQ ID NO:2, having protease activity; and
iii. a polypeptide having at least 80% sequence identity to SEQ ID NO:3, having protease activity.

6. The animal feed additive according to any of paragraphs 1 to 5, wherein the granule prepared by an extrusion process is a granule comprising
i. the polypeptide having protease activity and having at least 70% sequence identity to SEQ ID NO:1;
ii. a hydrophobic substance; and
iii. a solid carrier.

7. The animal feed additive according to paragraph 6, wherein the hydrophobic substance is selected from an oil and wax, such as selected from the group consisting of hydrogenated castor oil, hydrogenated palm kernel oil, hydrogenated rapeseed oil, hydrogenated palm oil, a blend of hydrogenated and unhydrogenated vegetable oil, 12-hydroxystearic acid, microcrystalline wax such as Cerit HOT, and high-melting paraffin waxes such as Mekon White.

8. The animal feed additive according to paragraph 6, wherein the solid carrier is selected from the group consisting of an absorbent and/or adsorbent material, such as a plant-based absorbent or a mineral sourced absorbent.

9. The animal feed additive according to any of paragraphs 1 to 5, wherein the granule prepared by a spray-drying process further comprises a carbohydrate.

10. The animal feed additive according to any of paragraphs 1 to 5, wherein the granule prepared by a spray-drying process comprises the step (a) preparing a spray liquid comprising a polypeptide having protease activity and having at least 70% sequence identity to the polypeptide of SEQ ID NO: 1 and a carbohydrate.

11. The animal feed additive according to paragraphic wherein the spray-drying process comprises the step
(a) preparing a spray liquid comprising a polypeptide having protease activity and having at least 70% sequence identity to the polypeptide of SEQ ID NO: 1 and a carbohydrate; and
(b) spraying the spray liquid in a spray tower.

12. The animal feed additive according to paragraph 11, wherein the spray tower has an inlet temperature of 100-200° C. and/or a product temperature of 50-80° C.

13. The animal feed additive according to paragraphs 9 to 12, wherein the carbohydrate is selected from the group consisting of lactose, sucrose, mannitol, α-cyclodextrin and dextrin, preferably dextrin.

14. The animal feed additive according to any of paragraphs 1 to 5 and 9 to 13, wherein the granule is prepared by a spray-drying process and wherein the granule has a water content of less than 7%.

15. The animal feed additive according to any of paragraphs 1 to 5 and 9 to 14, wherein the granule comprises or consists of a protease, dextrin and water.

16. The animal feed additive according to any of paragraphs 1 to 5, wherein enzyme granule comprises a salt core and a protease-containing layer, wherein the protease is a polypeptide having protease activity and having at least 70% sequence identity with SEQ ID NO:1.

17. The animal feed additive according to paragraph 16, wherein the salt core is a sodium sulfate or sodium chloride core, or a combination thereof.

18. The animal feed additive according to any of paragraph 16 to 17, wherein the enzyme granule has a particle size of 100-2000 micrometers, preferably 200-1500 micrometers, more preferably 300-1200 micrometers.

19. The animal feed additive according to any of paragraph 16 to 18, wherein the granule has a water content of less than 5%.

20. The animal feed additive according to any of paragraph 16 to 19, wherein the granule is prepared by a method comprising the steps of (a) preparing a salt core; and (b) distributing an protease liquid onto the salt core.

21. The animal feed additive according to paragraph 20, wherein the protease liquid is distributed onto the sodium sulfate or sodium chloride core by spray.

22. The animal feed additive according to any of paragraph 16 to 21, wherein the enzyme granule is prepared in a fluid bed apparatus.

23. The animal feed additive according to any of paragraphs 1 to 5 comprising a polypeptide having protease activity and having at least 70% sequence identity to SEQ ID NO:1 said polypeptide in granule or granulate prepared by a high-shear granulation process.

24. The animal feed additive according to paragraph 23 wherein the high-shear granulation process comprises the following steps
A. forming a powder mixture by combining at least
   i. cellulose or a derivative thereof
   ii. a binder; and
   iii. optionally a filler; and
B. adding a liquid phase granulating agent
   wherein the polypeptide having protease activity is added to either the powder mixture or to the liquid phase granulating agent.

25. An enzyme granule comprising a salt core, such as a sodium sulfate or sodium chloride core, and a protease containing layer, wherein the protease is a polypeptide having protease activity and having at least 70% sequence identity with SEQ ID NO: 1.

26. The enzyme granule according to paragraph 25, wherein the core is sodium sulfate core.

27. The enzyme granule according to paragraph 25 or 26, wherein the protease is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1;
(b) a variant of the polypeptide of SEQ ID NO: 1, comprising a substitution, deletion, and/or insertion of one or more (e.g. several) positions; and
(c) a fragment of the polypeptide of (a) or (b) that has protease activity.

28. The enzyme granule according to any of paragraphs 25 to 27, wherein the polypeptide comprises or consists of SEQ ID NO: 1.

29. The enzyme granule according to any of paragraphs 25 to 28, wherein the enzyme granule has a particle size of 100-2000 micrometers, preferably 200-1500 micrometers, more preferably 300-1200 micrometers.

30. The enzyme granule according to any of paragraphs 25 to 29, wherein the granule has a water content of less than 5%.

31. A method of producing an enzyme granule, comprising
(a) preparing a salt core such as a sodium sulfate, or sodium chloride core; and
(b) distributing a protease liquid to the sodium sulfate, or sodium chloride core.

32. The method according to paragraph 31, wherein the protease liquid is distributed onto the sodium sulfate or sodium chloride core by spray.

33. The method according to paragraph 31 or 32, wherein the enzyme granule is prepared in a fluid bed apparatus.

34. An animal feed, comprising the enzyme granule according to any of paragraphs 25 to 30.

35. Use of the enzyme granule according to any of paragraphs 25 to 30 in an animal feed.

36. A granule comprising a polypeptide having protease activity and having at least 70% sequence identity to the polypeptide of SEQ ID NO: 1; said granule prepared by a spray-drying process.

37. The granule according to paragraph 36 further comprising a carbohydrate.

38. The granule according to paragraph 36 or 37, wherein the carbohydrate is selected from the group consisting of lactose, sucrose, mannitol, α-cyclodextrin and dextrin, preferably dextrin.

39. The granule according to paragraph 36 to 38, wherein the protease is selected from the group consisting of:
(a) a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1;
(b) a variant of the polypeptide of SEQ ID NO: 1, comprising a substitution, deletion, and/or insertion of one or more (e.g. several) positions; and
(c) a fragment of the polypeptide of (a) or (b) that has protease activity.

40. The granule according to any of paragraphs 36 to 39, wherein the polypeptide comprises or consists of SEQ ID NO: 1.

41. The granule according to any of paragraphs 36 to 40, produced by a spray drying process comprising (a) preparing a spray liquid comprising a polypeptide having protease activity and having at least 70% sequence identity to the polypeptide of SEQ ID NO: 1; and a carbohydrate.

42. The granule according to any of paragraphs 36 to 41, wherein the granule has a water content of less than 7%.

43. The granule according to any of paragraphs 36 to 42, wherein the granule comprises or consists of protease, dextrin and water.

44. A method of producing an enzyme granule, comprising
(a) preparing a spray liquid comprising a polypeptide having protease activity and having at least 70% sequence identity to the polypeptide of SEQ ID NO: 1; and a carbohydrate; and
(b) spraying the spray liquid in a spray tower.

45. The method according to paragraph 44, wherein the carbohydrate is selected from the group consisting of lactose, sucrose, mannitol, α-cyclodextrin and dextrin, preferably dextrin.

1. The method according to paragraph 44 to 45, wherein the spray tower has an inlet temperature of 100-200° C. and/or a product temperature of 50-80° C.

46. An enzyme granule for use in animal feed, said granule defined by any of paragraphs 44 to 45.

47. An animal feed comprising the granule according to any of the paragraphs 44 to 45.

48. Use of the enzyme granule according to any of paragraphs 44 to 46 in an animal feed.

49. A method of producing an enzyme granule, comprising
(a) preparing a spray liquid comprising a polypeptide having protease activity and having at least 70% sequence identity to the polypeptide of SEQ ID NO: 1; and a carbohydrate; and
(b) spraying the spray liquid in a spray tower.

50. The method according to paragraph 49, wherein the carbohydrate is selected from the group consisting of lactose, sucrose, mannitol, α-cyclodextrin and dextrin, preferably dextrin.

51. The method according to paragraph 49, wherein the spray tower has an inlet temperature of 100-200° C. and/or a product temperature of 50-80° C.

52. An enzyme granulate said granulate prepared by a method comprising a high-shear granulation process, said granulate comprising a polypeptide having protease activity, said polypeptide having at least 70% sequence identity with a polypeptide of SEQ ID NO:1.

53. An enzyme granulate according to paragraph 52, further comprising at least one binder and cellulose or a derivative thereof.

54. The enzyme granulate of paragraph 52 wherein said high-shear granulation process comprises
forming a powder mixture by combining at least
   i. cellulose or a derivative thereof
   ii. optionally a binder; and
   iii. optionally a filler; and
adding a liquid phase granulating agent
wherein the polypeptide having protease activity is added to either the powder mixture or to the liquid phase granulating agent and wherein the at least one binder is added to either the powder mixture or to the liquid phase granulating agent or both.

55. The enzyme granulate of paragraphs 52 or 53 wherein said high-shear granulation process comprises
forming a powder mixture by combining at least
   i. cellulose or a derivative thereof
   ii. a binder; and
   iii. optionally a filler; and
adding a liquid phase granulating agent
wherein the polypeptide having protease activity is added to either the powder mixture or to the liquid phase granulating agent.

56. The enzyme granulate according to any of paragraphs 52 to 55 comprising a binder selected from the group consisting of polyvinyl pyrrolidone, titanium dioxide, dextrins, polyvinylalcohol, cellulose and cellulose derivatives, such as hydroxypropyl cellulose, methyl cellulose or carboxymethyl cellulose (CMC).

57. The enzyme granulate according to any of paragraphs 52 to 56, comprising a filler selected from the group consisting of any component which does not interfere with the granulating process, such as inorganic salts.

58. The enzyme granulate according to any of paragraphs 52 to 57, comprising cellulose or a derivative thereof in fibrous form.

59. The enzyme granulate according to any of paragraphs 52 to 58, wherein the liquid phase granulating agent is selected from the group consisting of a waxy substance and/or water or aqueous solution.

60. The enzyme granulate according to paragraph 59 wherein the waxy substance is selected from the group consisting of polyglycols, fatty alcohols, ethoxylated fatty alcohols, higher fatty acids, mono-, di- and triglycerolesters of higher fatty acids, such as glycerolmonostearate, alkylarylethoxylates, and coconut monoethanolamide.

61. The enzyme granulates according to paragraph 59 wherein the liquid phase granulating agent is water.

62. The enzyme granulates according to any of paragraphs 52 to 58, substantially free from a wax coating or a salt coating.

63. The enzyme granulates of any of paragraphs 52 to 62, has a density from 0.35 to 0.8, such as 0.37 to 0.7, such as 0.40 to 0.6.

64. An animal feed additive comprising the enzyme granulate of any of paragraphs 52 to 63.

65. An animal feed comprising the enzyme granulate of any of paragraphs 1 to 12.

66. A method of preparing a granulate comprising a granulate comprising a high-shear granulation, said high-shear granulation process comprising
forming a powder mixture by combining at least
   i. cellulose or a derivative thereof
   ii. optionally a binder; and
   iii. optionally a filler; and
adding a liquid phase granulating agent
wherein the polypeptide having protease activity is added to either the powder mixture or to the liquid phase granulating agent and wherein the at least one binder is added to either the powder mixture or to the liquid phase granulating agent or both;

wherein said polypeptide having protease activity is a polypeptide having at least 70% sequence identity to SEQ ID NO:1,

67. A method of preparing a granule by an extrusion process, said method comprising (a) combining a polypeptide having protease activity, a solid carrier, optionally water, and a meltable hydrophobic substance to provide a combined product; (b) optionally applying sufficient heat to the combined product to allow the hydrophobic substance to melt; (c) extruding the product of step (b); and (d) allowing the extruded product of step (c) to dry and cool or actively drying and cooling the extruded product of step (c) to provide the thermostable enzyme product, wherein the polypeptide having protease activity has at least 70% sequence identity to SEQ ID NO: 1, namely at least 75% sequence identity to the polypeptide of RONOZYME® ProAct.

68. The method according to paragraph 67 for preparing a thermostable enzyme product for use in the manufacture of animal feed comprising (a) combining an enzyme, a solid carrier and a meltable hydrophobic substance to provide a combined product; (b1) reducing the moisture content by applying heat to the combined product and (b2) melting the hydrophobic substance; and (c) cooling the combined product to provide the thermostable enzyme product, wherein the thermostable enzyme is the polypeptide having protease activity and having at least 70% sequence identity to SEQ ID NO: 1.

69. The method according to embodiment 67 for preparing a thermostable enzyme product for use in the manufacture of animal feed comprising (a) combining an enzyme, a solid carrier, a meltable hydrophobic substance to provide a combined product and optionally additional water to form a suitable paste; (b) optionally applying sufficient heat to the combined product to allow the hydrophobic substance to melt; (c) extruding the product of step (b); and (d) drying and cooling the extruded product of step (c) to provide the thermostable enzyme product. In one aspect of this embodiment, the meltable hydrophobic substance is added in step (a) as solid flakes or as a pre-melted molten liquid.

70. The method according to embodiment 67 for preparing a thermostable enzyme product for use in the manufacture of animal feed comprising (a) combining a polypeptide having protease activity and having at least 70% sequence identity to SEQ ID NO: 1, a solid carrier, a meltable hydrophobic substance to provide a combined product and optionally additional water to form a suitable paste; (b) melting the hydrophobic substance, or allowing the hydrophobic to melt, optionally by applying heat to the combined product; (c) extruding the product of step (b); and (d) optionally drying and cooling the extruded product of step (c) to provide the thermostable enzyme product.

71. The method according to any of embodiments 67 to 71, wherein the extruding step is at a temperature of 60° C. to 120° C., such as 70° C. to 110° C., such as 70° C. to 100° C., such as 80° C. to 100° C.

72. The method according to any of embodiments 67 to 71, wherein the extruding step further comprises a liquid binder and is performed at a temperature of 25° C. to 70° C., such as 30° C. to 70° C., such as of 30° C. to 60° C., such as 25° C. to 55° C., such as 30° C. to 55° C.

73. Animal feed granules prepared by a method according to any of embodiments 67 to 73.

### EXAMPLES

### Example 1. Extrusion With Hydrogenated Castor Oil

### General Process

The preparation of solid protease-containing extrusion products is a granule extrusion process wherein all components are combined in a mixing process with a suitable amount of water to act as a mixing agent for the components. The resulting wet mixture is then extruded through a suitable extrusion apparatus to produce wet granulates. These wet granulates are then further processed to shape the granules, and then dried to a suitable moisture content.

SEQ ID NO: 1 is mixed about 1:10 wt/wt with wheat flour. While these two components were mixing, molten HCO (about same weight as the protein) is poured into the mixture. Water is then added and the entire premix is blended for an additional approximately a minute. After this time the premix is extruded through a 0.5 mm to 1 mm, such as 0.8 mm screen and the wet strands are broken up and then rounded in spheroniser. The 0.8 mm extrudate produces approximately 0.8 mm spherical or rounded pellets. After the spheronising process, the wet granules are transferred to a dryer and dried for 20 minutes at approximately 50-90° C. The dried product is sieved through about 1.2 mm and 0.4-0.5 mm sieves. The fraction retained on the 0.4-0.5 mm sieve is a suitable extrudate for use as a feed additive

Other samples are prepared which contained varying ratios of hydrogenated castor oil to protein ratio or wheat flour to protein ratio.

### Example 2. Extrusion With a Blend of Hydrogenated and Unhydrogenated Vegetable Oil

For these experiments, SEQ ID NO: 1 was mixed with wheat flour in an about 1:10 weight ratio. While these two components are mixing, a blend of hydrogenated and unhydrogenated vegetable oil (PB3) (about in the same weight as the protein) is added as a soft solid and blended into the wheat flour. Water is then added (about in the same weight as the protein) and the entire premix is blended for about a minute. After this time the premix is extruded through a 0.8 mm screen and the wet strands are broken up and then rounded in a spheroniser. After the spheronising process, the wet granules are dried for 20 minutes at approximately 50-70° C. The dried product is sieved through 1.2 mm and 0.6 mm sieves. The fraction retained on the 0.6 mm sieve is a suitable extrudate for use as a feed additive

### Example 3. Incorporation of a Solid Hydrogenated Castor Oil by an Extrusion Process

Instead of adding molten HCO or softened PB3, a method was designed to incorporate the solid form of a meltable hydrophobic substance into the extrusion process. HCO flakes are mixed with wheat flour in a 1:10 weight ratio. The polypeptide of SEQ ID NO: 1 is then added with mixing. Water iss added and the entire premix is blended for an additional minute. The premix is extruded through 0.8 mm screen and the wet strands are broken up then rounded in a spheroniser. The wet granules are dried for 20 minutes at 50 to 75 C. The dried product is sieved through 1.2 mm and 0.5 mm sieves. The granules retained by the 0.5 mm are used for the preparative of the animal feed.

### Example 4. Incorporation of Solid Akoflake FSR by an Extrusion Process

In another experiment, Akoflake FSR, a fully hardened rapeseed oil, was used as in Example 3.

### Example 5: Protease Activity in Extruded Pellets

For the polypeptide having protease activity to be effective as an animal feed, the activity of the protease must be retained at effective levels during the pelletization process. Typically, the feed pellets are extruded through high-temperature nozzles prior to drying and subsequent feeding. The extrusion process described above result in a free-flowing product that exhibited an increased degree of enzyme protection. The granules produced in above are used to manufacture animal feed through a conventional pelleting process. Activity of the polypeptide having protease activity in the extrusion pellet is comparable to the activity of the same polypeptide of Ronozyme Proact.

### Example 6: Granule Prepared by a Spray-Drying Process: Enzyme Layer on Salt Core

| | |
|---|---|
| A protease derived from Nocardiopsis sp. NRRL 18262 (an acid-stable protease) | SEQ ID NO: 1 herein; also shown as SEQ ID NO: 1 of WO 2001/058276 |

A spray fluid having the following composition
80000 g SEQ ID NO:1 concentrate
20000 g Dextrin W80
60000 g Water
is sprayed in a spray tower with an inlet temperature of 160°
C and a product temperature of 65C. The spray dried granules have a water content less than 7 %.

### Example 7: Enzyme Layer on Salt Core

| | |
|---|---|
| A protease derived from Nocardiopsis sp. NRRL 18262 (an acid-stable protease) | SEQ ID NO: 1 herein; also shown as SEQ ID NO: 1 of WO 2001/058276 |

4000 g Na₂SO₄ cores, were loaded into a Glatt Procell (GF3, bottom spray) fluid bed;

A granulation fluid consisting of
1200 g A protease derived from Nocardiopsis sp. NRRL 18262 concentrate
were sprayed on top of the Na₂SO₄ cores at a product temperature of 65° C.
The granulated was dried to a water content of less than 5% and sifted to obtain a product with the particle size between 300 and 1180 micrometers.

### Example 8: High Shear Granulation

### High Shear Mixer Without Coating

A powder mixture with the following composition

| | |
|---|---|
| 1200 g | Cellulose, Arbocel BC200 |
| 600 g | Dextrin W80 |
| 1200 g | CaCO₃ |
| 10552 g | Ground Na₂SO₄ |

was granulated in a Lödige mixer FM 50 with a granulation fluid consisting of 3544 g Ronozyme ProAct (SEQ ID NO:1) enzyme concentrate

The granulation was carried out as described in U.S. Pat. 4,106,991, example 1.

The granulated was dried in a fluid bed dryer to a water content of less than 1%.

### Example 9: High Shear Granulation II

20-30 percent (25 percent) Ronozyme ProAct (SEQ ID NO:1), 10 percent cellulose fibres, 1 percent binder: PVP K 30 1. Powder components; 7.5 kg ground proteolytic enzyme Ronozyme ProAct, 0.6 kg titanium dioxide 3.0 kg cellulose powder-CEPO S 20 (The Swedish cellulose powder and Wood Flour Mills Ltd.) 18.6 kg ground sodium chloride

2. The above components are mixed on the Lodige mixer FM 130 D I Z with a rotating speed of the mixer of 160 rpm and with a revolution speed of a single cross knife granulating device of 3000 rpm for 1 minute.

3. Thereafter wetting is performed with a 3-6 percent, such as 4.5 percent aqueous solution of polyvinylpyrrolidone (PVP K 30) during continuous mixing with both mixing-aggregate and granulating device.

4. After spraying of the binder solution, the moist mixture is further exposed to the compacting action of the granulating device for 7-10 minutes.

The rotating speed on the mixing aggregate is kept on 160 rpm and on the granulating device on 3000 rpm.

### Example 10 : Pelleting Stability Model

The purpose of these tests is to evaluate the stability of a novel formulation of the protease found in the commercial product RONOZYME® ProAct CT and the stability of commercially available proteases. The pelleting stability model tests are performed at 95° C. with a 90 seconds incubation applying parameters used in industrial pelleting process. Experiments are run in triplicates and a mean average is reported. Enzyme activity and recovery is measured using a spectrophotometric assay based on the Suc-AAPP-pNA substrate (pNA assay). In this assay, the enzyme product is mixed with the substrate in a buffer at pH 7.0 and 37° C. for 15 minutes and kinetics activity are measured monitoring the product reaction absorbance at 405 nm.

Residual activity of the protease products after steam treatment is evaluated using the following assay: 250 mg of each enzyme product is dispensed into aluminum cups. The stress steam incubation is performed in a closed styropor container with the inner dimensions 27 × 18 × 20 cm. One liter of boiling water is poured into a steam generator. The steam is transferred from the steam generator into the box. The samples are placed on a plate and inserted into the box through a drawer when the temperature of 95° C. is reached. The temperature in the box is monitored using a thermometer mounted in the lid of the container. The incubation proceeds for 90 seconds from the moment the samples are inserted into the box. Immediately after the incubation the samples are cooled down on ice, re-suspended in 0.1 M Acetate buffer, 5 mM Ca++, pH 5.0 and the protease activity is measured using the pNA assay described above. Each enzyme product is compared to a similar sample that has not undergone the steam treatment test and residual activity is evaluated as the ratio between the activity of the steam-box treated samples and the control samples. The results of the experiments are reported in Tables 1 to 4 and show that the protease in the novel formulation has a stability similar to the one of the ProAct CT commercial product and a higher stability compared to other commercial products.

In the large-scale production of Ronozyme® ProAct CT is the activity approximately 90% so the 90 second test is best predictive in this steam stability model he results show that for the cheaper formulations of the protease, namely for an extruded enzyme pellet, a granule comprising a salt core and a protease-containing layer, and a granulate prepared by a method comprising a high-shear granulation process, in the 90 second steam stability test at 95° C., comparable stability was achieved to the Ronozyme® ProAct CT commercial product

### Micro-Granulation Formulation of SEQ ID NO:1(ProAct): Granule Comprising a Salt Core and A Protease Containing Layer (ProAct)

TABLE 1

Residual activity after pelleting stability model test of different protease formulation and commercial products. Enzyme products were stressed with a temperature 95° C. for 90 s.

| Sample | Residual activity after pelleting stability model stress (%) |
|---|---|
| granule comprising a salt core and a protease-containing layer (SEQ ID NO:1) | 89 |
| ProAct CT | 88 |
| TP100 (DuPont) | 62 |
| Protease (Huaxin) | 28 |
| Neutral protease (VTR) | 0 |
| 511RA-2 (SunHy) | 0 |
| Acid protease (VTR) | 0 |

### High-Shear Granulation Formulation of SEQ ID NO:1(ProAct)

TABLE 2

Residual activity after pelleting stability model test of different protease formulation and commercial products. Enzyme products were stressed with a temperature 95° C. for 90 s.

| Sample | Residual activity after pelleting stability model stress (%) |
|---|---|
| High shear formulation of SEQ ID NO:1 | 90 |
| ProAct CT | 88 |
| TP100 (DuPont) | 62 |
| Protease (Huaxin) | 28 |
| Neutral protease (VTR) | 0 |
| 511RA-2 (SunHy) | 0 |
| Acid protease (VTR) | 0 |

### Extrusion Formulation of of SEQ ID NO:1(ProAct)

TABLE 3

Residual activity after pelleting stability model test of different protease formulation and commercial products. Enzyme products were stressed with a temperature 95° C. for 90 s

| Sample | Residual activity after pelleting stability model stress (%) |
|---|---|
| Extrusion formulation of SEQ ID NO:1 | 83 |
| ProAct CT | 88 |
| TP100 (DuPont) | 62 |
| Protease (Huaxin) | 28 |
| Neutral protease (VTR) | 0 |
| 511RA-2 (SunHy) | 0 |
| Acid protease (VTR) | 0 |

### Example 11 - Pelleting Stability Model

### Pilot Granulation, Temperature Stability Tests and Activity Analysis

The purpose of these tests is to evaluate the stability of a novel formulation of the protease found in the commercial product RONOZYME® ProAct CT and the stability of the commercially available protease ProAct. The pelleting stability model tests are performed at 95° C. (to simulate a temperature applied in industrial pelleting process) with a 5 minutes incubation. Experiments are run in triplicates and a mean average is reported. Enzyme activity and recovery is measured using a spectrophotometric assay based on the Suc- Ala-Ala-Pro-Phe-pNA substrate (pNA assay). In this assay, the enzyme product is mixed with the substrate in a buffer at pH 7.0 and 37° C. for 15 minutes and kinetics activity are measured monitoring the product reaction absorbance at 405 nm.

Residual activity of the protease products after temperature treatment is evaluated using the following assay: 25 mg of each enzyme product is dispensed into a 0.2 mL tube (thin-walled 8 tube strips, Thermo Scientific); 5 µL of deionized water is added to the lid of each tube in order to simulate the humidity of the pelleting process. The tubes are placed into a PCR equipment (GeneAmp PCR system 9700 Perkin Elmer) and incubated for 5 minutes at 95° C. Immediately after the incubation the samples are cooled down on ice, re-suspended in 5 ml of 0.1 M Acetate buffer, 5 mM Ca++, pH 5.0 and the protease activity is measured using the pNA assay described above. Each enzyme product is compared to a similar sample that has not undergone the temperature treatment test and residual activity is evaluated as the ratio between the activity of the steam-box treated samples and the control samples. The results of the experiments are reported in Table 1 and show that the protease in the novel formulation has a stability similar to the one of the ProAct CT commercial product.

TABLE 4

Residual activity after pelleting stability model test of a novel protease formulation and commercial products. Enzyme products were stressed with a temperature 95° C. for 5 minutes

| Sample | Residual activity after pelleting stability model stress (%) |
|---|---|
| ProAct Spray drying formulation | 92 |
| ProAct CT | 96 |

### Example 12: Determination of SDS-PAGE Purity of Protease Samples

The SDS-PAGE purity of the protease samples was determined by the following procedure:

40µl protease solution (A₂₈₀ concentration = 0.025) was mixed with 10µl 50%(w/v) TCA (trichloroacetic acid) in an Eppendorf tube on ice. After half an hour on ice the tube was centrifuged (5 minutes, 0° C., 14.000 x g) and the supernatant was carefully removed. 20µl SDS-PAGE sample buffer (200µl Tris-Glycine SDS Sample Buffer (2x) (125 mM Tris/HCl, pH 6.8, 4%(w/v) SDS, 50 ppm bromophenol blue, 20%(v/v) Glycerol, LC2676 from NOVEX™) + 160µl dist. water + 20µl ß-mercaptoethanol + 20µl 3 M unbuffered Tris Base (Sigma T-1503) was added to the precipitate and the tube was boiled for 3 minutes. The tube was centrifuged shortly and 10µl sample was applied to a 4-20% gradient Tris-Glycine precast gel from NOVEX™ (polyacrylamide gradient gel based on the Laemmli chemistry but without SDS in the gel, (Laemmli, U.K., (1970) Nature, vol. 227, pp. 680-685), EC60255). The electrophoresis was performed with Tris-Glycine running buffer (2.9 g Tris Base, 14.4 g Glycine, 1.0 g SDS, distilled water to 1 liter) in both buffer reservoirs at a 150 V constant voltage until the bromophenol blue tracking dye had reached the bottom of the gel. After electrophoresis, the gel was rinsed 3 times, 5 minutes each, with 100 ml of distilled water by gentle shaking. The gel was then gently shaked with Gelcode^{®} Blue Stain Reagent (colloidal Comassie G-250 product from PIERCE, PIERCE cat. No. 24592) for one hour and washed by gentle shaking for 8 to 16 hours with distilled water with several changes of distilled water. Finally, the gel was dried between 2 pieces of cellophane. Dried gels were scanned with a Arcus II scanner from AGFA equipped with Fotolook 95 v2.08 software and imported to the image evaluation software CREAM™ for Windows (catalogue nos. 990001 and 990005, Kem-En-Tec, Denmark) by the File/Acquire command with the following settings (of Fotolook 95 v2.08): Original=Reflective, Mode=Color RGB, Scan resolution=240 ppi, Output resolution=120Ipi, Scale factor=100%, Range=Histogram with Global selection and Min=0 and Max=215, ToneCurve=None, Sharpness=None, Descreen=None and Flavor=None, thereby producing an *.img picture file of the SDS-PAGE gel, which was used for evaluation in CREAM™. The *.img picture file was evaluated with the menu command Analysis/1-D. Two scan lines were placed on the *.img picture file with the Lane Place Tool: A Sample scan line and a Background scan line. The Sample scan line was placed in the middle of a sample lane (with the protease in question) from just below the application slot to just above the position of the Bromphenol blue tracking dye. The Background scan line was placed parallel to the Sample scan line, but at a position in the pictured SDS-PAGE gel where no sample was applied, start and endpoints for the Background scan line were perpendicular to the start and endpoints of the Sample scan line. The Background scan line represents the true background of the gel. The width and shape of the scan lines were not adjusted. The intensity along the scan lines where now recorded with the 1-D/Scan menu command with Medium sensitivity. Using the 1-D/Editor menu command, the Background scan was subtracted from the Sample scan. Then the 1-D/Results menu command was selected and the Area % of the protease peak, as calculated by the CREAM™ software, was used as the SDS-PAGE purity of the proteases.

All the protease samples had an SDS-PAGE purity of above 95%.

### Example 13: pH-Activity Assay

Suc-AAPF-pNA (Sigma® S-7388) was used for obtaining pH-activity profiles.

Assay buffer: 100 mM succinic acid (Merck 1.00682), 100 mM HEPES (Sigma H-3375), 100 mM CHES (Sigma C-2885), 100 mM CABS (Sigma C-5580), 1 mM CaCl₂, 150 mM KCI, 0.01% Triton® X-100, adjusted to pH-values 3.0, 4.0, 5.0, 6.0, 7.0, 8.0, 9.0, 10.0, or 11.0 with HCl or NaOH.

Assay temperature: 25° C.

A 300µl protease sample (diluted in 0.01% Triton® X-100) was mixed with 1.5 ml of the assay buffer at the respective pH value, bringing the pH of the mixture to the pH of the assay buffer. The reaction was started by adding 1.5 ml pNA substrate (50 mg dissolved in 1.0 ml DMSO and further diluted 45x with 0.01% Triton® X-100) and, after mixing, the increase in A₄₀₅ was monitored by a spectrophotometer as a measurement of the protease activity at the pH in question. The assay was repeated with the assay buffer at the other pH values, and the activity measurements were plotted as relative activity against pH. The relative activities were normalized with the highest activity (pH-optimum), i.e. setting activity at pH-optimum to 1, or to 100%. The protease samples were diluted to ensure that all activity measurements fell within the linear part of the dose-response curve for the assay.

### Example 14: pH-Stability Assay

Suc-AAPF-pNA (Sigma® S-7388) was used for obtaining pH-stability profiles.

Assay buffer: 100 mM succinic acid, 100 mM HEPES, 100 mM CHES, 100 mM CABS, 1 mM CaCl₂, 150 mM KCI, 0.01% Triton® X-100 adjusted to pH-values 2.0, 2.5, 3.0, 3.5, 4.0, 4.5, 5.0, 6.0, 7.0, 8.0, 9.0, 10.0 or 11.0 with HCl or NaOH.

Each protease sample (in 1 mM succinic acid, 2 mM CaCl₂, 100 mM NaCl, pH 6.0 and with an A₂₈₀ absorption > 10) was diluted in the assay buffer at each pH value tested to A₂₈₀ = 1.0. The diluted protease samples were incubated for 2 hours at 37° C. After incubation, protease samples were diluted in 100 mM succinic acid, 100 mM HEPES, 100 mM CHES, 100 mM CABS, 1 mM CaCl₂, 150 mM KCI, 0.01% Triton® X-100, pH 9.0, bringing the pH of all samples to pH 9.0.

In the following activity measurement, the temperature was 25° C. 300µl diluted protease sample was mixed with 1.5 ml of the pH 9.0 assay buffer and the activity reaction was started by adding 1.5 ml pNA substrate (50 mg dissolved in 1.0 ml DMSO and further diluted 45x with 0.01% Triton® X-100) and, after mixing, the increase in A₄₀₅ was monitored by a spectrophotometer as a measurement of the (residual) protease activity. The 37° C. incubation was performed at the different pH-values and the activity measurements were plotted as residual activities against pH.

The residual activities were normalized with the activity of a parallel incubation (control), where the protease was diluted to A₂₈₀ = 1.0 in the assay buffer at pH 9.0 and incubated for 2 hours at 5° C. before activity measurement as the other incubations. The protease samples were diluted prior to the activity measurement in order to ensure that all activity measurements fell within the linear part of the dose-response curve for the assay.

### Example 15 Temperature-Activity Assay

Protazyme AK tablets were used for obtaining temperature profiles. Protazyme AK tablets are azurine dyed crosslinked casein prepared as tablets by Megazyme.

Assay buffer: 100 mM succinic acid, 100 mM HEPES, 100 mM CHES, 100 mM CABS, 1 mM CaCl₂, 150 mM KCI, 0.01% Triton^{®} X-100 adjusted to pH 9.0 with NaOH.

A Protazyme AK tablet was suspended in 2.0ml 0.01% Triton^{®} X-100 by gentle stirring. 500µl of this suspension and 500µl assay buffer were mixed in an Eppendorf tube and placed on ice. 20µl protease sample (diluted in 0.01% Triton X-100) was added. The assay was initiated by transferring the Eppendorf tube to an Eppendorf thermomixer, which was set to the assay temperature. The tube was incubated for 15 minutes on the Eppendorf thermomixer at its highest shaking rate. By transferring the tube back to the ice bath, the assay incubation was stopped. The tube was centrifuged in an ice-cold centrifuge for a few minutes and the A₆₅₀ of the supernatant was read by a spectrophotometer. A buffer blind was included in the assay (instead of enzyme). A₆₅₀(protease) - A₆₅₀(blind) was a measurement of protease activity. The assay was performed at different temperatures and the activity measurements were plotted as relative activities against incubation temperature. The relative activities were normalized with the highest activity (temperature optimum). The protease samples were diluted to ensure that all activity measurements fell within the near linear part of the dose-response curve for the assay.

An overview of the activity optima (pH- and temperature activity) is seen in Table 5 and a detailed comparison of the pH-stability data for the proteases at acidic pH-values is seen in Table 6.

TABLE 5

pH- and temperature optima of the protease

| Protease | pH-optimum (pNA-substrate) | Temperature-optimum at pH 9.0 (Protazyme AK) |
|---|---|---|
| Nocardiopsis sp. NRRL 18262 | 10 | 70°C |

TABLE 6

pH-stability of the protease, between pH 2.0 and 5.0

| Protease | pH 2.0 | pH 2.5 | pH 3.0 | pH 3.5 | pH 4.0 | pH 4.5 | pH 5.0 |
|---|---|---|---|---|---|---|---|
| Nocardiopsis sp. NRRL 18262 | 0.779 | 1.000 | 1.029 | 0.983 | 0.991 | 1.019 | 1.004 |

### Example 16: Absorption Purity of Purified Protease Samples

### Determination of A₂₈₀/A₂₆₀ Ratio

The A₂₈₀/A₂₆₀ ratio of purified protease samples was determined as follows.

A₂₆₀ means the absorption of a protease sample at 260 nm in a 1 cm path length cuvette relative to a buffer blank. A₂₈₀ means the absorption of the same protease sample at 280 nm in a 1 cm path length cuvette relative to a buffer blank.

Samples of the purified proteases were diluted in buffer until the A₂₈₀ reading of the spectrophotometer is within the linear part of its response curve. The A₂₈₀/A₂₆₀ ratio was determined from the readings: For Nocardiopsis sp. NRRL 18262 1.83.

### Example 17: Ability of Protease Derived From Nocardiopsis Sp. NRRL 18262 to Degrade Insoluble Parts of Soy Bean Meal (SBM)

The protease from Nocardiopsis sp. NRRL 18262 was tested for its ability to make the insoluble/indigestible parts of SBM accessible to digestive enzymes and/or added exogeneous enzymes.

Its performance was compared to two aspartate proteases, Protease I and Protease II, prepared as described in WO 95/02044. This document also discloses their use in feed. Protease I is an Aspergillopepsin II type of protease, and Protease II an Aspergillopepsin I type of protease (both aspartate proteases, i.e. non-subtilisin proteases) from Aspergillus aculeatus (reference being made to Handbook of Proteolytic Enzymes referred to above).

The test substrate, the so-called soy remnant, was produced in a process which mimics the digestive tract of mono-gastric animals, including a pepsin treatment at pH 2, and a pancreatin treatment at pH 7.

In the pancreatin treatment step a range of commercial enzymes was added in high dosages in order to degrade the SBM components that are accessible to existing commercial enzymes.

The following enzymes, all commercially available from Novozymes A/S, Denmark, were added:

ALCALASE™ 2.4 L, NEUTRASE™ 0.5 L, FLAVOURZYME™ 1000 L, ENERGEX™ L, BIOFEED™ Plus L, PHYTASE NOVO™ L. The SBM used was a standard 48% protein SBM for feed, which had been pelletised.

After the treatment only 5% of the total protein was left in the resulting soy remnant.

### FITC Labelling Protocol

The remnant was subsequently labelled with FITC (Molecular Probes, F-143) as follows: Soy remnant (25 g wet, ~ 5 g dry) was suspended in 100 ml 0.1 M carbonate buffer, pH 9 and stirred 1 hour at 40° C. The suspension was cooled to room temperature and treated with fluorescein 5-isothiocyanate (FITC) over night in the dark. Non-coupled probe was removed by ultrafiltration (10.000 Mw cut-off).

### FITC-Assay

The FITC-labelled soy remnant was used for testing the ability of the proteases to degrade the soy remnant using the following assay: 0.4 ml protease sample (with A₂₈₀ = 0.1) was mixed with 0.4 ml FITC-soy remnant (suspension of 10 mg/ml in 0.2 M sodium-phosphate buffer pH 6.5) at 37° C., and the relative fluorescence units (RFU 485/535 nm; excitation/monitoring wave length) measured after 0 hours, and after 22 hours incubation. Before determination of the RFU, samples were centrifuged for 1 min at 20.000 x G and 250 micro-liter supernatant was transferred to a black micro-titer tray. Measurements were performed using a VICTOR 1420 Multilabel counter (In vitro, Denmark). RFU is generally described by lain D. Johnson in: Introduction to Fluorescence Techniques, Handbook of Fluorescent Probes and Research Chemicals, Molecular Probes, Richard P. Haugland, 6^{th} edition, 1996 (ISBN 0-9652240-0-7).

A blind sample was prepared by adding 0.4 ml buffer instead of enzyme sample.

$$\begin{matrix}{\text{RFU}}_{\text{sample}} = Δ {\text{RFU}}_{\text{sample}} - Δ {\text{RFU}}_{\text{blind,}} \text{where} Δ \text{RFU =} \\ \text{RFU} \left(22 \text{hours}\right) - \text{RFU} \left(0 \text{hours}\right)\end{matrix}$$

The resulting FITC values (RFUₛₐₘₚₗₑ values) are shown in Table 7 below. The FITC values are generally with an error margin of +/- 20.000. Contrary to Protease I and Protease II, the protease derived from Nocardiopsis sp. NRRL 18262 degraded the soy remnant to a significant extent.

TABLE 7

Ability of proteases to degrade soy remnant

| Protease | FITC (+/-20000) |
|---|---|
| Nocardiopsis sp. NRRL 18262 | 92900 |
| Protease I | -9200 |
| Protease II | -1200 |

### Example 18: In Vitro Testing of the Protease Derived From Nocardiopsis Sp. NRRL 18262

The protease derived from Nocardiopsis sp. NRRL 18262 was tested, together with a protease derived from Bacillus sp. NCIMB 40484 (“PD498,” prepared as described in Example 1 of WO93/24623), and together with FLAVOURZYME™, a protease-containing enzyme preparation from Aspergillus oryzae (commercially available from Novozymes A/S, Bagsvaerd, Denmark), for its ability to solubilise maize-SBM (maize-Soy Bean Meal) proteins in an in vitro digestion system (simulating digestion in monogastric animals). For the blank treatments, maize-SBM was incubated in the absence of exogenous proteases.

### Outline of in Vitro Model

| | |
|---|---|
| Components added to flask | Time course (min) |
| 10 g maize-SBM (60:40) + HCl/pepsin (3000U/g diet) + protease (0.1 mg enzyme protein/g diet or 3.3 mg FLAVOURZYME™ /g diet), T=40° C., pH=3.0 | t=0 |
| NaOH, T=40° C., pH 6 | t=60 |
| NaHCO₃/pancreatin (8.0 mg/g diet), T=40° C., pH 6-7 | t=80 |
| Stop incubation and take samples, T=0° C. | t=320 |

### Substrates

10 g maize-SBM diet with a ratio maize-SBM of 6:4 (w/w) was used. The protein content was 43% (w/w) in SBM and 8.2% (w/w) in maize meal. The total amount of protein in 10 g maize-SBM diet was 2.21 g.

### Digestive Enzymes

Pepsin (Sigma P-7000; 539 U/mg, solid), pancreatin (Sigma P-7545; 8xU.S.P. (US Pharmacopeia)).

### Enzyme Protein Determinations

The amount of protease enzyme protein is calculated on the basis of the A₂₈₀ values and the amino acid sequences (amino acid compositions) using the principles outlined in S.C.Gill & P.H. von Hippel, Analytical Biochemistry 182, 319-326, (1989).

### Experimental Procedure for in Vitro Model

1. 10 g of substrate is weighed into a 100 ml flask.
2. At time 0 min, 46 ml HCl (0.1 M) containing pepsin (3000 U/g diet) and 1 ml of protease (0.1 mg enzyme protein/g diet, except for FLAVOURZYME™: 3.3 mg/g diet) are added to the flask while mixing. The flask is incubated at 40° C.
3. At time 20-25 min, pH is measured.
4. At time 45 min, 16 ml of H₂O is added.
5. At time 60 min, 7 ml of NaOH (0.4 M) is added.
6. At time 80 min, 5 ml of NaHCO₃ (1 M) containing pancreatin (8.0 mg/g diet) is added.
7. At time 90 min, pH is measured.
8. At time 300 min, pH is measured.
9. At time 320 min, aliquots of 30 ml are removed and centrifuged (10000 × g, 10 min, 0° C.).
10. Total soluble protein in supernatants is determined.

### Protein Determination

Supernatants are analysed for protein content using the Kjeldahl method (determination of % nitrogen; A.O.A.C. (1984) Official Methods of Analysis 14^{th} ed. Association of Official Analytical Chemists, Washington DC).

### Calculations

For all samples, in vitro protein solubility was calculated using the equations below.

Amount of protein in diet sample: 22.1% of 10 g = 2.21 g

If all the protein were solubilised in the 75 ml of liquid, the protein concentration in the supernatant would be: 2.21 g/75 ml ≈ 2.95%.

Note that the supernatants also include the digestive and exogenous enzymes. In order to determine the solubility, the protein contribution from the digestive and exogenous enzymes should be subtracted from the protein concentrations in the supernatants whenever possible.

% protein from the pancreatin (X mg/g diet) and pepsin (Y U/g diet) = ((Xmg pancreatin/g diet × 10 g diet × 0.69 × 100%)/(1000 mg/g × 75 g)) + ((YU pepsin/g diet × 10 g diet x 0.57 × 100%)/(539 U/mg × 1000 mg/g × 75 g)),

where 0.69 and 0.57 refer to the protein contents in the pancreatin and pepsin preparations used (i.e. 69% of the pancreatin, and 57% of the pepsin is protein as determined by the Kjeldahl method referred to above).

% protein from exogenous enzymes (Z mg EP/g diet)= (Z mg EP/g diet × 10 g diet × 100%)/(1000 mg/g × 75 g)

% protein corrected in supernatant = % protein in supernatant as analysed - (% protein from digestive enzymes + % protein from exogenous enzymes)

Protein solubilisation (%) = (% protein corrected in supernatant x 100%)/2.95 %

The results below show that the protease derived from Nocardiopsis sp. NRRL 18262 has a significantly better effect on protein solubilisation as compared to the blank, and as compared to the Bacillus sp. NCIMB 40484 protease.

| Enzyme | Solubilised P (% of total) | SD | n |
|---|---|---|---|
| Blind (no exogenous enzymes) | 73.8^{c} | 0.87 | 10 |
| + the protease derived from Nocardiopsis sp. NRRL 18262 | 77.5^{a} | 0.50 | 10 |
| + the protease derived from Bacillus sp. NCIMB 40484 | 75.6^{b} | 1.52 | 5 |
| + FLAVOURZYME™ | 74.1^{c} | 0.23 | 4 |

^{a,b,c} Values within a column not sharing a common superscript letter are significantly different, P<0.05. SD is standard deviation; n is the number of observations.

### Example 19: Degradation of the Lectin SBA and the Soybean Bowman-Birk and Kunitz Inhibitors The Ability of the Proteases from Nocardiopsis sp. NRRL 18262 and Bacillus sp. NCIMB 40484 to Hydrolyse Soybean Agglutinin (SBA) and the Soy Bowman-Birk and Kunitz TRYPSIN Inhibitors was Tested

Pure SBA (Fluka 61763), Bowman-Birk Inhibitor (Sigma T-9777) or Kunitz Inhibitor (Trypsin Inhibitor from soybean, Boehringer Mannheim 109886) was incubated with the protease for 2 hours, 37° C., at pH 6.5 (protease: anti-nutritional factor = 1:10, based on A₂₈₀). Incubation buffer: 50 mM dimethyl glutaric acid, 150 mM NaCl, 1 mM CaCl₂, 0.01% Triton X-100, pH 6.5.

The ability of the proteases to degrade SBA and the protease inhibitors was estimated from the disappearance of the native SBA or trypsin inhibitor bands and appearance of low molecular weight degradation products on SDS-PAGE gels. Gels were stained with Coomassie blue and band intensity determined by scanning.

The results, as % of anti-nutritional factor degraded, are shown in Table 8 below.

It is contemplated that the ability to degrade the anti-nutritional factors in soy can also be estimated by applying the Western technique with antibodies against SBA, Bowman-Birk Inhibitor or Kunitz Inhibitor after incubation of soybean meal with the candidate proteases (see WO98/56260).

TABLE 8

| Protease derived from | SBA | Bowman-Birk Inhibitor | Kunitz Inhibitor |
|---|---|---|---|
| Nocardiopsis sp. NRRL 18262 | 75 | 25 | 100 |
| Bacillus sp. NCIMB 40484 | 21 | 41 | 100 |

### Example 20: Effects of Acid-Stable Nocardiopsis Proteases on the Growth Performance of Broiler Chickens

The trial is carried out in accordance with the official French instructions for experiments with live animals. Day-old broiler chickens (‘Ross PM3’), separated by sex, are supplied by a commercial hatchery.

The chickens are housed in wire-floored battery cages, which are kept in an environmentally controlled room. Feed and tap water is provided ad libitum.

On day 8, the chickens are divided by weight into groups of 6 birds, which are allocated to either the control treatment, receiving the experimental diet without enzymes, or to the enzyme treatment, receiving the experimental diet supplemented with 100 mg enzyme protein of the protease per kg feed.

Each treatment is replicated with 12 groups, 6 groups of each sex. The groups are weighed on days 8 and 29. The feed consumption of the intermediate period is determined and body weight gain and feed conversion ratio are calculated.

The experimental diet is based on maize starch and soybean meal (44% crude protein) as main ingredients (Table 5). The feed is pelleted (die configuration: 3 × 20 mm) at about 70° C. An appropriate amount of the protease is diluted in a fixed quantity of water and sprayed onto the pelleted feed. For the control treatment, adequate amounts of water are used to handle the treatments in the same way.

For the statistical evaluation, a two factorial analysis of variance (factors: treatment and sex) is carried out, using the GLM procedure of the SAS package (SAS Institute Inc., 1985). Where significant treatments effects (p < 0.05) are indicated, the differences between treatment means are analysed with the Duncan test. An improved weight gain, and/or an improved feed conversion, and/or improved nutritive value of soybean meal is expected (taking into consideration that maize starch is a highly digestible ingredient).

### References

EEC : Directive de la Commission du 9 avril 1986 fixant la méthode de calcul de la valeur energetique des aliments composes destines a la volaille. Journal Officiel des Communautes Europeennes, L130, 53 - 54

SAS Institute Inc. (1985): SAS® User’s Guide, Version 5 Edition. Cary NC

TABLE 5

Composition of the experimental diet

| Inqredients (%): | |
|---|---|
| Maize starch | 45.80 |
| Soybean meal 44 ¹ | 44.40 |
| Tallow | 3.20 |
| Soybean oil | 1.00 |
| DL-Methionine | 0.18 |
| MCP | 0.76 |
| Salt | 0.05 |
| Binder | 1.00 |
| Vitamin and mineral premix | 3.55 |
| Avatec® 15% CC ² | 0.06 |
| Analysed content: | |
| Crude protein (%) | 19.3 |
| ME, N-corrected (MJ/kg) ³ | 12.2 |
| Crude fat (%) | 5.3 |

¹ analysed content: 90.6% dry matter, 45.3% crude protein, 2.0% crude fat, 4.9% crude fibre
² corresponded to 90 mg lasalocid-Na / kg feed as anticoccidial
³ calculated on the basis of analysed nutrients content (EC-equation; EEC, 1986)

### Supplier of Feed Ingredients

Maize starch: Roquettes Freres, F-62136 Lestrem, France
Soybean meal 44: Rekasan GmbH, D-07338 Kaulsdorf, Germany
Tallow: Fondoirs Gachot SA, F-67100 Strasbourg, France
Soybean oil: Ewoco Sarl, F-68970 Guemar, France
DL-Methionine: Produit Roche SA, F-92521 Neuilly-sur-Seine, France
MCP: Brenntag Lorraine, F-54200 Toul, France
Salt: Minoterie Moderne, F-68560 Hirsingue, France
Binder: Minoterie Moderne, F-68560 Hirsingue, France
Premix (AM vol chair NS 4231): Agrobase, F-01007 Bourg-en-Bresse, France
Avatec: Produit Roche SA, F-92521 Neuilly-sur-Seine, France

### Example 21: Premix and Diets for Turkey and Salmonids Supplemented With Acid-Stable Nocardiopsis Protease

A premix of the following composition is prepared (content per kilo):

| | | |
|---|---|---|
| 5000000 | IE | Vitamin A |
| 1000000 | IE | Vitamin D3 |
| 13333 | mg | Vitamin E |
| 1000 | mg | Vitamin K3 |
| 750 | mg | Vitamin B1 |
| 2500 | mg | Vitamin B2 |
| 1500 | mg | Vitamin B6 |
| 7666 | mg | Vitamin B12 |
| 12333 | mg | Niacin |
| 33333 | mg | Biotin |
| 300 | mg | Folic Acid |
| 3000 | mg | Ca-D-Panthothenate |
| 1666 | mg | Cu |
| 16666 mg | Fe | |
| 16666 | mg | Zn |
| 23333 | mg | Mn |
| 133 | mg | Co |
| 66 | mg | I |
| 66 | mg | Se |
| 5.8 | % | Calcium |

To this premix the protease from Nocardiopsis sp. NRRL 18262 is added (prepared as described in Example 2), in an amount corresponding to 10 g protease enzyme protein/kg.

Pelleted turkey starter and grower diets with a composition as shown in the below table (on the basis of Leeson and Summers, 1997 but recalculated without meat meal by using the AGROSOFT®, optimisation program) and with 100 mg protease enzyme protein per kg are prepared as follows:

Milled maize, Soybean meal, Fish-meal and Vegetable fat are mixed in a cascade mixer. Limestone, calcium phosphate and salt are added, together with the above premix in an amount of 10 g/kg diet, followed by mixing. The resulting mixture is pelleted (steam conditioning followed by the pelleting step).

| Ingredient | Starter diet, g/kg | Grower, g/kg | Finisher |
|---|---|---|---|
| Maize | 454.4 | 612.5 | 781.0 |
| Soybean meal | 391 | 279 | 61.7 |
| Fish meal | 70 | 29.9 | 70 |
| Vegetable fat | 21 | 21 | 46 |
| Limestone | 19 | 16.9 | 9 |
| Calcium phosphate | 30 | 25.9 | 16.8 |
| Salt (NaCl) | 2 | 2 | 2 |
| Vitamin and mineral premix | 10 | 10 | 10 |
| Lysine | 1.3 | 1.49 | |
| Methionine | 1.3 | 1.3 | 3.6 |
| Calculated nutrients | | | |
| Crude protein g/kg | 279 | 213 | 152 |
| Metabolisable energy MJ/kg | 12.3 | 12.7 | 14.1 |
| Calcium, g/kg | 15.8 | 12.7 | 9 |
| Available Phosphorus, g/kg | 8.2 | 6.4 | 4.6 |
| Lysine, g/kg | 17.6 | 12.8 | 7.5 |
| Methionine, g/kg | 6.1 | 4.9 | 6.9 |

Two diets for Salmonids are also prepared, as generally outlined above. The actual compositions are indicated in the Table below (compiled from Refstie et al (1998), Aquaculture, vol. 162, p.301-302). The estimated nutrient content is recalculated by using the Agrosoft® feed optimisation program.

The protease derived from Nocardiopsis alba, prepared as described in Example 2, is added to the diets in an amount corresponding to 100 mg protease enzyme protein per kg.

| Ingredient | Conventional diet with fish meal | Alternative diet with soybean meal |
|---|---|---|
| Wheat | 245.3 | 75.2 |
| Fish meal | 505.0 | 310.0 |
| Soybean meal | | 339.0 |
| Fish oil | 185.0 | 200.0 |
| DL-Methionine | 13.9 | 23.0 |
| Mono-Calcium phosphate | | 2.0 |
| Vitamin and Mineral premix + pellet binder and astaxanthin | 50.8 | 50.8 |
| Calculated nutrients (fresh weight basis) | | |
| Crude protein g/kg | 401 | 415 |
| Crude fat g/kg | 232 | 247 |
| Metabolisable energy MJ/kg | 16.9 | 16.5 |
| Calcium, g/kg | 13.9 | 9.8 |
| Phosphorus, g/kg | 10.8 | 9.0 |
| Lysine, g/kg | 27.7 | 26.7 |
| Methionine, g/kg | 24.4 | 31.6 |

### Example 22: Determination of Purity of Protease-Containing Enzyme Products

The purity of protease-containing enzyme products, e.g. protease preparations such as commercial multi-component enzyme products, can be determined by a method based on the fractionation of the protease-containing enzyme product on a size-exclusion column. Size-exclusion chromatography, also known as gel filtration chromatography, is based on a porous gel matrix (packed in a column) with a distribution of pore sizes comparable in size to the protein molecules to be separated. Relatively small protein molecules can diffuse into the gel from the surrounding solution, whereas larger molecules will be prevented by their size from diffusing into the gel to the same degree. As a result, protein molecules are separated according to their size with larger molecules eluting from the column before smaller ones.

### Protein Concentration Assay

The protein concentration in protease-containing enzyme products is determined with a BCA protein assay kit from PIERCE (identical to PIERCE cat. No.23225). The sodium salt of Bicinchoninic acid (BCA) is a stable, water-soluble compound capable of forming an intense purple complex with cuprous ions (Cu¹⁺) in an alkaline environment. The BCA reagent forms the basis of the BCA protein assay kit capable of monitoring cuprous ions produced in the reaction of protein with alkaline Cu²⁺ (Biuret reaction). The colour produced from this reaction is stable and increases in a proportional fashion with increasing protein concentrations (Smith, P.K., et al. (1985), Analytical Biochemistry, vol. 150, pp. 76-85). The BCA working solution is made by mixing 50 parts of reagent A with 1 part reagent B (Reagent A is PIERCE cat. No. 23223, contains BCA and tartrate in an alkaline carbonate buffer; reagent B is PIERCE cat. No. 23224, contains 4% CuSO₄*5H₂O). 300µl sample is mixed with 3.0 ml BCA working solution. After 30 minutes at 37° C., the sample is cooled to room temperature and A₄₉₀ is read as a measure of the protein concentration in the sample. Dilutions of Bovine serum albumin (PIERCE cat. No. 23209) are included in the assay as a standard.

### Sample Pre-Treatment

If the protease-containing enzyme product is a solid, the product is first dissolved/suspended in 20 volumes of 100 mM H₃BO₃, 10 mM 3,3′-dimethylglutaric acid, 2 mM CaCl₂, pH 6 (Buffer A) for at least 15 minutes at 5° C., and if the enzyme at this stage is a suspension, the suspension is filtered through a 0.45µ filter to give a clear solution. The solution is from this point treated as a liquid protease-containing enzyme product.

If the protease-containing enzyme product is a liquid, the product is first dialysed in a 6-8000 Da cut-off SpectraPor dialysis tube (cat.no. 132 670 from Spectrum Medical Industries) against 100 volumes of Buffer A + 150 mM NaCl (Buffer B) for at least 5 hours at 5° C., to remove formulation chemicals that could give liquid protease-containing enzyme products a high viscosity, which is detrimental to the size-exclusion chromatography.

The dialysed protease-containing enzyme product is filtered through a 0.45µ filter if a precipitate was formed during the dialysis. The protein concentration in the dialysed enzyme product is determined with the above-described protein concentration assay and the enzyme product is diluted with Buffer B, to give a sample ready for size-exclusion chromatography with a protein concentration of 5 mg/ml. If the enzyme product has a lower than 5 mg/ml protein concentration after dialysis, it is used as is.

### Size-Exclusion Chromatography

A 300ml HiLoad26/60 Superdex75pg column (Amersham Pharmacia Biotech) is equilibrated in Buffer B (Flow: 1 ml/min). 1.0 ml of the protease-containing enzyme sample is applied to the column and the column is eluted with Buffer B (Flow: 1 ml/min). 2.0 ml fractions are collected from the outlet of the column, until all of the applied sample have eluted from the column. The collected fractions are analysed for protein content (see above Protein concentration assay) and for protease activity by appropriate assays. An example of an appropriate assay is the Suc-AAPF-pNA assay Other appropriate assays are e.g. the CPU assay and the Protazyme AK assay.The conditions, e.g. pH, for the protease activity assays are adjusted to measure as many proteases in the fractionated sample as possible. The conditions of the assays referred to above are examples of suitable conditions. Other suitable conditions are mentioned above in the section dealing with measurement of protease activity. A protein peak with activity in one or more of the protease assays is defined as a protease peak. The purity of a protease peak is calculated as the protein amount in the peak divided with the total protein amount in all identified protease peaks. The purity of a protease-containing enzyme product is calculated as the amount of protein in the protease peak divided with the protein amount in all identified protease peaks using the above procedure.

1. An animal feed additive comprising a polypeptide in a formulation selected from the group consisting of:
i. a granule prepared by an extrusion process;
ii. a granule prepared by a spray-drying process;
iii. a granule comprising a salt core and a layer that contains said polypeptide; and
iv. a granule prepared by a high-shear granulation process,
wherein the polypeptide exhibits protease activity and comprises an amino acid sequence having at least 70% sequence identity with the amino acid sequence set forth herein as SEQ ID NO: 1.

2. The animal feed additive of claim 1, wherein the polypeptide is obtained or obtainable from Nocardiopsis sp. NRRL 18262.

3. The animal feed additive of claim 1, wherein the polypeptide is in a granule prepared by an extrusion process, said granule comprising:
i. a hydrophobic substance; and
ii. a solid carrier.

4. The animal feed additive of claim 1, wherein the polypeptide is in a granule prepared by a spray-drying process, said spray-drying process comprising:
(a) preparing a spray liquid comprising the polypeptide and a carbohydrate; and
(b) spraying the spray liquid in a spray tower.

5. The animal feed additive of claim 4, wherein the carbohydrate is dextrin.

6. The animal feed additive of claim 1, wherein the polypeptide is in a granule that comprises a salt core and a layer that contains said polypeptide.

7. The animal feed additive of claim 6, wherein the salt core comprises sodium sulfate and/or sodium chloride.

8. The animal feed additive of claim 6, wherein the granule has a particle size of 100-2000 micrometers.

9. The animal feed additive of claim 6, wherein the granule was prepared in a fluid bed apparatus.

10. The animal feed additive of claim 1, wherein the polypeptide is in a granule prepared by a high-shear granulation process.

11. The animal feed additive of claim 10, wherein the high-shear granulation process comprises the following steps:
A. forming a powder mixture by combining at least
i. cellulose or a derivative thereof
ii. a binder; and
iii. optionally a filler; and
B. adding a liquid phase granulating agent
wherein the polypeptide is added to either the powder mixture or to the liquid phase granulating agent.

12. A granule comprising a salt core and a polypeptide-containing layer, said polypeptide-containing layer comprising a polypeptide that exhibits protease activity and that comprises an amino acid sequence having at least 70% sequence identity with the amino acid sequence set forth herein as SEQ ID NO: 1.

13. A granule comprising a polypeptide that exhibits protease activity and that comprises an amino acid sequence having at least 70% sequence identity with the amino acid sequence set forth herein as SEQ ID NO: 1.

14. A method of preparing a granule comprising a polypeptide having protease activity and having at least 70% sequence identity to the polypeptide of SEQ ID NO: 1, said method comprising a formulation process selected from the group consisting of
i. extrusion;
ii. spray-drying;
iii. spraying or wetting a salt core with a protease-containing liquid; and
iv. high-shear granulation.


SEQUENCE LISTING (3 sequences)

SEQ ID NO: 1 - protein, 564 aa, Nocardiopsis sp.
ALAASPILEILEGLYGLYLEUALATYRTHRMETGLYGLYARGCYSSERVALGLYPHEALAALATHRASNALAALAGLYGLNPROGLYPHEVALTHRALAGLYHISCYSGLYARGVALGLYTHRGLNVALTHRILEGLYASNGLYARGGLYVALPHEGLUGLNSERVALPHEPROGLYASNASPALAALAPHEVALARGGLYTHRSERASNPHETHRLEUTHRASNLEUVALSERARGTYRASNTHRGLYGLYTYRALATHRVALALAGLYHISASNGLNALAPROILEGLYSERSERVALCYSARGSERGLYSERTHRTHRGLYTRPHISCYSGLYTHRILEGLNALAARGGLYGLNSERVALSERTYRPROGLUGLYTHRVALTHRASNMETTHRARGTHRTHRVALCYSALAGLUPROGLYASPSERGLYGLYSERTYRILESERGLYTHRGLNALAGLNGLYVALTHRSERGLYGLYSERGLYASNCYSARGTHRGLYGLYTHRTHRPHETYRGLNGLUVALTHRPROMETVALASNSERTRPGLYVALARGLEUARGTHR

SEQ ID NO: 2 - protein, 188 aa, synthetic construct
ADIIGGLAYTIGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 3 - protein, 388 aa, synthetic construct
MKKPLGKIVASTALLISVAFSSSIASAAPAPVPQTPVADDSAASMTEALKRDLDLTSAEAEELLSAQEAAIETDAEATEAAGEAYGGSLFDTETLELTVLVTDASAVEAVEATGAQATVVSHGTEGLTEVVEDLNGAEVPESVLGWYPDVESDTVVVEVLEGSDADVAALADAGVDSSSVRVEEAEEAPQVYADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRTQSHVQSAP